(19) United States
(12) Patent Application Publication
(10) Pub. No.: US 2024/0295566 A1
(43) Pub. Date: Sep. 5, 2024

(54) METHOD FOR DIAGNOSING AND TREATING METABOLIC DISORDERS

(71) Applicant: Centre for Chinese Herbal Medicine Drug Development Limited, Hong Kong (CN)

(72) Inventors: Zhaoxiang BIAN, Hong Kong (CN); Hoi Leong Xavier WONG, Hong Kong (CN); Lixiang ZHAI, Hong Kong (CN); Johnson Yiu-Nam LAU, Hong Kong (CN); Chengyuan LIN, Hong Kong (CN); Haitao XIAO, Hong Kong (CN)

(21) Appl. No.: 18/169,934

(22) Filed: Feb. 16, 2023

(51) Int. Cl. G01N 33/68; A61K 31/40; A61K 31/44; A61P 3/00; G01N 33/569

(52) U.S. Cl. CPC G01N 33/6893; A61K 31/40; A61K 31/44; A61P 3/00; G01N 33/56911; G01N 2800/04

(57) ABSTRACT

Provided herein is a method of diagnosing a metabolic disorder in a subject involving determining the amount of at least one of Ruminococcus gnavus, phenethylamine, and tryptamine in a fecal sample obtained from the subject. Also provided is a method of treating a metabolic disorder in a subject in need thereof involving the administration of a therapeutically effective amount of a trace amine-associated receptor 1 inhibitor.

### REFERENCE TO SEQUENCE LISTING

The Sequence Listing identified as Sequence_Listing_P24905US00.xml; Size: 5,944 bytes; and Date of Creation: Jan. 18, 2023, filed herewith, is hereby incorporated by reference in its entirety.

### TECHNICAL FIELD

The present invention disclosure relates to methods of diagnosis and/or treatment of a metabolic disorder.

### BACKGROUND

The gut microbiome has been extensively studied in the past decades for its association with metabolic disorders including obesity, insulin resistance, and type 2 diabetes (T2D). Dysbiosis of gut microbiota has been causatively linked with the development of metabolic disorders, yet the pathogenic role of gut microbes at the species level remains elusive. Changes in gut microbiota composition in patients with metabolic disorders have been shown to induce changes in the content of gut-microbial products including lipopolysaccharides, short-chain fatty acids, bile acids, trimethylamine N-oxide and imidazole propionate, and these effects are widely recognized for their beneficial or detrimental effects on glucose tolerance in both animal and human studies. In addition to these microbial metabolites, dietary amino acids including aromatic and branched-chain amino acids can also be catabolized by the gut microbiota into numerous metabolites that may affect the metabolic health of hosts. Despite the strong association between altered gut microbiota, diets and metabolic disorders, the pathogenetic mechanisms caused by altered gut microbes and microbe-derived products in the development of metabolic disorders are still far from the whole landscape.

Despite advances in understanding the pathogenetic mechanisms caused by altered gut microbes and microbe-derived products, there still exists a need for improved methods of treating and/or diagnosing metabolic disorders, such as insulin resistance.

### SUMMARY

The present disclosure relates to the diagnosis and treatment of insulin resistance. Here, the inventors identified tryptamine and phenethylamine, metabolites derived from gut-microbial catabolism of dietary aromatic amino acids, as major contributing factors for gut dysbiosis-induced insulin resistance. Monoassociation of human gut bacterium Ruminococcus gnavus, both a metabolic syndrome-associated gut microbe and a major producer of tryptamine and phenethylamine, impaired insulin sensitivity and induced glucose intolerance in germ-free mice. Tryptamine and phenethylamine were positively associated with glucose intolerance in both patients with type 2 diabetes (T2D) and monkeys with spontaneous diabetes. Administration of tryptamine and phenethylamine directly impaired insulin signaling in major metabolic tissues of normal mice and monkeys, which was mediated by the trace amine-associated receptor 1 (TAAR1)-extracellular signal-regulated kinase (ERK) signaling axis. Inhibition of TAAR1 alleviated insulin resistance induced by the colonization of R. gnavus in antibiotics-treated mice. It was further demonstrated that tryptamine and phenethylamine are remarkably reduced in T2D subjects under dietary fiber intervention and negatively correlated with improvement of insulin sensitivity. These findings suggest a causal role for tryptamine/phenethylamine-producers in the development of insulin resistance, and highlight the tryptamine and phenethylamine/TAAR1 signaling axis as a potential therapeutic target for the management of metabolic disorders, such as insulin resistance, pre-diabetes, and diabetes.

In a first aspect, provided herein is a method of treating a metabolic disorder in a subject in need thereof, the method comprising: providing a fecal sample obtained from the subject; determining the amount of one or more markers selected from the group consisting of phenethylamine, tryptamine, and Ruminococcus gnavus in the fecal sample; determining based on the amount of the one or more markers in the fecal sample that the subject has the metabolic disorder; and administering a therapeutically effective amount of a trace amine-associated receptor 1 (TAAR1) inhibitor to the subject.

In certain embodiments, the metabolic disorder is selected from the group consisting of insulin resistance, glucose intolerance, hyperglycemia, hyperlipidemia, pre-diabetes, type 2 diabetes, and combinations thereof.

In certain embodiments, the metabolic disorder comprises insulin resistance.

In certain embodiments, the TAAR1 inhibitor selectively binds TAAR1.

In certain embodiments, the TAAR1 inhibitor is a small molecule.

In certain embodiments, the TAAR1 inhibitor is a compound of Formula 1:

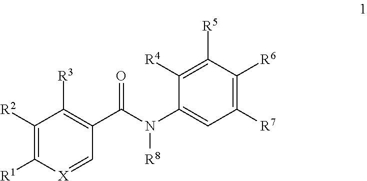

or a pharmaceutically acceptable salt thereof, wherein
n is 0, 1, 2 or 3;
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;
X is —C(R⁹)═ or —N═;
R⁹ is hydrogen, C₁-C₆ alkoxy, NO₂, or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

In certain embodiments, the TAAR1 inhibitor is N-(3-ethoxy-phenyl)-4-pyrrolidin-1-yl-3-trifluoromethyl-benzamide.

In certain embodiments, the subject is a human, a non-human primate, a rodent, a canine, a feline, a bovine, or an equine.

In certain embodiments, the subject is a human.

In certain embodiments, the metabolic disorder comprises insulin resistance and the one or more markers comprises Ruminococcus gnavus.

In certain embodiments, the step of determining based on the amount of the one or more markers in the fecal sample that the subject has the metabolic disorder comprises comparing the amount of the one or more markers in the fecal sample with an average amount of the one or more markers in fecal samples obtained from healthy controls.

In certain embodiments, the amount of the one or more markers in the fecal sample obtained from the subject is higher than the average amount of the one or more markers in fecal samples obtained from healthy controls.

In a second aspect, provided herein is a method of treating insulin resistance in a subject in need thereof, the method comprising: providing a fecal sample obtained from the subject; determining the amount of Ruminococcus gnavus in the fecal sample; determining based on the amount of Ruminococcus gnavus in the fecal sample that the subject has the metabolic disorder; and administering a therapeutically effective amount of a trace amine-associated receptor 1 (TAAR1) inhibitor to the subject.

In certain embodiments, the TAAR1 inhibitor is a compound of Formula 1:

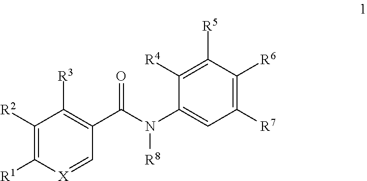

or a pharmaceutically acceptable salt thereof, wherein
n is 0, 1, 2 or 3;
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;
X is —C(R⁹)═ or —N═;
R⁹ is hydrogen, C₁-C₆ alkoxy, NO₂, or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

In certain embodiments, the TAAR1 inhibitor is N-(3-ethoxy-phenyl)-4-pyrrolidin-1-yl-3-trifluoromethyl-benzamide.

In a third aspect, provided herein is a method of diagnosing a metabolic disorder in a subject, the method comprising providing a fecal sample obtained from the subject; determining the amount of one or more markers selected from the group consisting of Ruminococcus gnavus, phenethylamine, and tryptamine in the fecal sample; determining based on the amount of the one or more markers in the fecal sample if the subject has the metabolic disorder.

In certain embodiments, the metabolic disorder is selected from the group consisting of insulin resistance, glucose intolerance, hyperglycemia, hyperlipidemia, pre-diabetes, type 2 diabetes, and combinations thereof.

In certain embodiments, the metabolic disorder comprises insulin resistance.

In certain embodiments, the one or more markers comprises Ruminococcus gnavus.

In certain embodiments, the method further comprises the step of administering a therapeutically effective amount of a trace amine-associated receptor 1 (TAAR1) inhibitor to the subject.

In certain embodiments, the TAAR1 inhibitor is a compound of Formula 1:

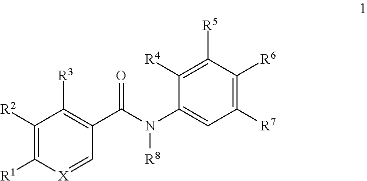

or a pharmaceutically acceptable salt thereof, wherein
n is 0, 1, 2 or 3;
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;
X is —C(R⁹)═ or —N═;
R⁹ is hydrogen, C₁-C₆ alkoxy, NO₂, or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

In certain embodiments, the TAAR1 inhibitor is N-(3-ethoxy-phenyl)-4-pyrrolidin-1-yl-3-trifluoromethyl-benzamide.

In certain embodiments, the one or more markers comprises Ruminococcus gnavus and the metabolic disorder comprises insulin resistance.

### BRIEF DESCRIPTION OF THE DRAWINGS

The above and other objects and features of the present invention will become apparent from the following description of the invention, when taken in conjunction with the accompanying drawings, in which:

FIG. 1 shows colonization of a tryptamine and phenethylamine producer R. gnavus impairs insulin sensitivity in germ-free mice. (A-B) OGTT and ITT indexes in germ-free mice following colonization of R. gnavus ATCC 29149 (n=6 per group). (C-D) Tryptamine and phenethylamine level in serum and fecal samples of germ-free mice following colonization of R. gnavus ATCC 29149 (n=6 per group). (E) Tryptamine and phenethylamine levels in fecal samples of normal mice from the control group (without antibiotics treatment, n=6) and the group treated with antibiotics mixture (n=12). (F-G) OGTT and ITT indexes in antibiotics-treated mice following colonization of either engineered L. casei TDC⁺ or L. casei WT (n=6 per group). (H) Tryptamine and phenethylamine levels in fecal samples of antibiotics-treated mice following colonization of either engineered L. casei TDC⁺ or L. casei WT for 3 days (n=6 per group). P values were determined by ordinary two-way ANOVA or Student's t-test. Data are presented as mean±S.D. See additional data in FIG. 7.

FIG. 2 shows tryptamine and phenethylamine positively correlate with glucose intolerance in patients with type 2 diabetes and monkeys with spontaneous diabetes. (A-B) Tryptamine and phenethylamine level in fecal samples of individuals with or without T2D (n=25 subjects with NGT, n=25 patients with T2D). (C-D) Spearman's correlation between fecal tryptamine/phenethylamine levels with FBG level in individuals with or without T2D (n=25 subjects with NGT, n=25 patients with T2D). (E-F) Tryptamine and phenethylamine level in serum and fecal samples of age-matched monkeys with or without pre-diabetes and diabetes (n=26/group). (G-H) Tryptamine and phenethylamine production in batch culture experiments using feces from monkeys with or without pre-diabetes and diabetes (n=26/group). (I-J) Spearman's correlation between fecal tryptamine/phenethylamine levels with FBG level in monkeys with or without pre-diabetes and diabetes (n=26/group). Differences of phenethylamine and tryptamine levels in serum and fecal samples were analyzed by one-tailed student t-tests. Data are presented as mean±S.D. See additional information in FIG. 8.

FIG. 3 shows tryptamine and phenethylamine impair insulin sensitivity in mice, monkeys and in vitro models. (A-B) OGTT and ITT in normal mice after treatment with tryptamine at indicated dosages (0.4 mg/kg, 2 mg/kg and 10 mg/kg) or control (1% DMSO in saline) by i.p. (n=6/group). * comparison between control group and tryptamine group (10 mg/kg). #comparison between control group and tryptamine-treated group (2 mg/kg). (C-D) OGTT and ITT in normal mice after treatment with phenethylamine at indicated dosages (1 mg/kg, 2 mg/kg and 5 mg/kg) or control (1% DMSO in saline) by i.p. (n=6/group). * comparison between control group and phenethylamine group (5 mg/kg). #comparison between control group and tryptamine-treated group (2 mg/kg). (E-F) Western blot (and quantification) of the effect of tryptamine treatment (10 mg/kg) by i.p. on AKT phosphorylation stimulated by insulin (1 U/kg) in WAT lysates, liver lysates and skeletal muscle lysates from mice. (n=3/group). (G-H) Western blot (and quantification) of the effect of phenethylamine treatment (5 mg/kg) by i.p. on AKT phosphorylation stimulated by insulin (1 U/kg) in WAT lysates and liver lysates from mice. (n=3/group). (I-J) Western blot (and quantification) of the effect of tryptamine treatment (25 μM) on insulin signaling stimulated by insulin (20 nM) in 3T3-L1 cells (n=3/group). (K-L) Western blot (and quantification) of the effect of phenethylamine treatment (25 μM) on insulin signaling stimulated by insulin (20 nM) in 3T3-L1 cells (n=3/group). Data are presented as mean±S.D. P values were determined by ordinary one-way ANOVA or Student's t-test. See additional information in FIG. 9.

FIG. 4 shows tryptamine and phenethylamine impair insulin signaling via ERK activation. (A-B) Western blot (and quantification) of the effect of tryptamine treatment (10 mg/kg) on ERK activation in WAT lysates, liver lysates and skeletal muscle lysates from mice (n=3/group). (C-D) OGTT and ITT indexes in mice after treatment with tryptamine (10 mg/kg), ERK inhibitor U0126 (20 mg/kg) or control (1% DMSO in saline) (n=6/group) by i.p. * comparison between control group and tryptamine group (10 mg/kg). #comparison between tryptamine group and tryptamine+ERK inhibitor (U0126) group. (E-F), Western blot (and quantification) of the effects of tryptamine (10 mg/kg) and ERK inhibitor U0126 treatment (20 mg/kg) on ERK activation and insulin (1 U/kg)-stimulated AKT activation in WAT lysates and liver lysates from mice (n=2-3/group) by i.p. (G-H) Western blot (and quantification) of the effect of tryptamine (25 μM) and ERK inhibitor U0126 on ERK activation in 3T3-L1 cells (n=3/group). (I-L) Western blot (and quantification) of the effects of tryptamine (25 μM) and ERK inhibitor U0126 treatment (20 μM) on ERK activation and insulin (20 nM)-stimulated AKT activation in 3T3-L1 cells (n=3/group). Data are presented as mean±S.D. P values were determined by ordinary one-way ANOVA or Student's t-test. See additional information in FIG. 10.

FIG. 5 shows tryptamine and phenethylamine weaken insulin signaling via TAAR1-ERK signaling axis. (A-B) OGTT and ITT indexes in mice after treatment with tryptamine (10 mg/kg), TAAR1 antagonist EPPTB (10 mg/kg) or control (1% DMSO in saline) by i.p. (n=6/group). * comparison between control group and tryptamine group (10 mg/kg). #comparison between tryptamine group and tryptamine+TAAR1 antagonist (EPPTB) group. (C-D) OGTT and ITT indexes in wildtype (WT) and Taar1 knock out (KO) mice after treatment with tryptamine (10 mg/kg) or control (1% DMSO in saline) by i.p. (n=6/group). * comparison between with and without tryptamine treatment (10 mg/kg) in WT mice. #comparison between tryptamine treatment in WT and Taar1 KO mice. (E-H) Western blot (and quantification) of the effects of tryptamine (10 mg/kg) and TAAR1 antagonist EPPTB (10 mg/kg) treatment on ERK activation and insulin (1 U/kg)-stimulated AKT activation in WAT lysates and liver lysates from mice (n=2-3/group). (I-J) OGTT and ITT indexes in antibiotics-treated mice following colonization by R. gnavus ATCC 29149 and treatment with TAAR1 antagonist EPPTB (10 mg/kg) by i.p. (n=6/group). * comparison between control group and tryptamine group (10 mg/kg). #comparison between tryptamine group and tryptamine+TAAR1 antagonist (EPPTB) group. (K-L) Western blot (and quantification) of effects of R. gnavus ATCC 29149 and TAAR1 antagonist EPPTB (10 mg/kg) on AKT activation stimulated by insulin (1 U/kg) in liver lysates from antibiotics-treated mice (n=3 per group). P values were determined by ordinary one-way ANOVA or Student's t-test. See additional information in FIG. 11.

FIG. 6 shows tryptamine and phenethylamine are negatively correlated with the improvement of insulin sensitivity in dietary fiber-treated patients with type 2 diabetes. (A) Tryptamine levels in fecal samples from T2D subjects in control group (U group; n=16) and high-fiber group (W group; n=27) (Day 0 and Day 84). (B-D) Spearman's correlation analysis between fecal tryptamine level and OGTT AUC, HOMA-IR and HbAlc indexes in T2D subjects consuming a high fiber diet (W group; n=27). (E) Phenethylamine levels in fecal samples from T2D subjects in control group (U group; n=16) and high-fiber group (W group; n=27) (Day 0 and Day 84). (F-H) Spearman's correlation analysis between fecal phenethylamine level and OGTT AUC, HOMA-IR and HbAlc indexes in T2D subjects consuming a high fiber diet (W group; n=27). Data are presented as mean±S.D. P values were determined by ordinary one-way ANOVA or paired student's t-test. See additional information in FIG. 12.

FIG. 7 shows colonization of a tryptamine and phenethylamine producer R. gnavus impairs insulin sensitivity in germ-free mice. (A) Spearman r and p-values (−log10p) plot against gut bacteria species abundances and TyG level in human participants (n=412). (B) Spearman's correlation between relative abundances of R. gnavus with TyG level in human participants (n=412). (C) Body weight changes in germ-free mice following colonization of R. gnavus ATCC 29149 (n=6 per group). (D-E) LC-MS chromatogram of tryptamine and phenethylamine level in MRS culture medium of L. casei TDC+ and L. casei vector control (WT). Differences in body weight changes were determined by two-way ANOVA. Data are presented as mean±S.D.

FIG. 8 shows tryptamine and phenethylamine positively correlate with glucose intolerance in patients with type 2 diabetes and monkeys with spontaneous diabetes. (A-B) Tryptophan and phenylalanine (substrates of tryptamine and phenethylamine) levels in fecal samples from individuals with or without T2D (n=25 subjects with NGT, n=25 patients with T2D). (C-D) Spearman's correlation between fecal tryptamine/phenethylamine levels with TyG level in healthy volunteers (n=89). (E-I) Age, body weight, FBG, HbA1c and TG levels in monkeys without or with pre-diabetic or diabetes (n=26/per group). (J) OGTT index in HFD-fed mice after treatment with fecal suspension from normal monkeys and diabetes monkeys once per day for 5 days (n=6/group). (K-M) Spearman's correlation between fecal/serum tryptamine levels with FBG and HbA1c level in monkeys with or without pre-diabetes and diabetes (n=26/group). (N-P) Spearman's correlation between fecal/serum phenethylamine levels with FBG and HbA1c level in monkeys with or without pre-diabetes and diabetes (n=26/group). Differences of phenethylamine and tryptamine levels in fecal and serum samples were analyzed by one-tailed student t-tests. Differences of age, body weight, FBG, HbA1c, TG and OGTT indexes in monkeys were analyzed by ordinary one-way ANOVA. Data are presented as mean±S.D.

FIG. 9 shows tryptamine and phenethylamine impair insulin sensitivity in mice, monkeys and in vitro models. (A-B) Serum TG level in mice (n=6/group) after treatment with tryptamine or phenethylamine at indicated dosages or control (1% DMSO in saline) by i.p. (C-D) IVGTT index and serum insulin levels in monkeys after treatment with tryptamine (10 mg/kg) or control (1% CMC-Na in water) (n=5/group). (E) Effect of tryptamine (5 μM, 10 μM and 25 μM) on glucose uptake stimulated by insulin (20 nM) in 3T3-L1 cells (n=3/group). (F) Western blot (and quantification) of tryptophan (100 μM) and indole acetic acid (100 μM) treatment (precursor and metabolite of tryptamine) on insulin signaling stimulated by insulin (20 nM) in 3T3-L1 cells (n=3/group). (G) Western blot (and quantification) of phenylalanine (100 μM) and phenylacetic acid (100 μM) treatment (precursor and metabolite of phenethylamine) on insulin signaling stimulated by insulin (20 nM) in 3T3-L1 cells (n=3/group). Data are presented as mean±S.D. P values were determined by ordinary one-way ANOVA or Student's t-test.

FIG. 10 shows tryptamine and phenethylamine impair insulin signaling via ERK activation. (A-B) Tryptamine and indole acetic acid levels in serum, WAT, liver, and skeletal muscle after administration of tryptamine (5 mg/kg) at indicated times (n=6). (C-D) OGTT and ITT indexes in mice after treatment of tryptamine (10 mg/kg), ERK inhibitor PD98509 (10 mg/kg) or control (1% DMSO in saline) (n=6/group). * Comparisons between control group and tryptamine group (10 mg/kg). #Comparisons between tryptamine group and tryptamine+ERK inhibitor (PD98059) group. (E-F) Western blot (and quantification) of the effect of phenethylamine (25 μM) and ERK inhibitor U0126 on ERK activation in 3T3-L1 cells (n=3/group). (G-H) Western blot (and quantification) of the effects of tryptamine (25 μM) and ERK inhibitor U0126 treatment (20 μM) on ERK activation and insulin (20 nM)-stimulated AKT activation in 3T3-L1 cells (n=3/group). Data are presented as mean±S.D. P values were determined by ordinary one-way ANOVA or Student's t-test.

FIG. 11 shows tryptamine and phenethylamine weaken insulin signaling via TAAR1-ERK signaling axis. (A-B) Western blot (and quantification) of the effects of tryptamine (25 μM) and TAAR1 antagonist EPPTB treatment (20 μM) on ERK activation in 3T3-L1 cells (n=3/group). (C-D) Western blot (and quantification) of the effects of tryptamine (25 μM) and TAAR1 antagonist EPPTB treatment (20 μM) on ERK activation and insulin (20 nM)-stimulated AKT activation in 3T3-L1 cells (n=3/group). (E-F) Western blot (and quantification) of the effects of phenethylamine (25 μM) and TAAR1 antagonist EPPTB treatment (20 μM) on ERK activation in 3T3-L1 cells (n=3/group). (G-H) Western blot (and quantification) of the effects of phenethylamine (25 μM) and TAAR1 antagonist EPPTB treatment (20 μM) on ERK activation and insulin (20 nM)-stimulated AKT activation in 3T3-L1 cells (n=3/group). (I) Western blot of tryptamine (25 μM), AhR antagonist CH223191 (10 μM), 5-HT2A receptor antagonist SR46349B (25 μM) and MDL100907 (20 μM) as well as 5-HT2B receptor antagonist SB204741 (20 μM) and LY266097 (20 μM) in 3T3-L1 cells (n=3/group). Data are presented as mean±S.D. P values were determined by ordinary one-way ANOVA or Student's t-test.

FIG. 12 shows tryptamine and phenethylamine are negatively correlated with the improvement of insulin sensitivity in dietary fiber-treated patients with type 2 diabetes. (A) Tyramine levels in fecal samples of individuals with or without type 2 diabetes (n=25 subjects with NGT, n=25 patients with T2D). (B) Tyramine levels in fecal samples of age-matched monkeys without or with pre-diabetes or diabetes (n=26/group). (C-D) Spearman's correlation analysis between relative abundances of Blautia hansenii with TyG level and fecal tryptamine/phenethylamine level in human participants (n=412). (E-F) Spearman's correlation analysis between relative abundances of Enterococcus faecalis with TyG level and fecal tryptamine/phenethylamine level in human participants (n=412). (G-H) Spearman's correlation analysis between relative abundances of Clostridium boltae with TyG level and fecal tryptamine/phenethylamine level in human participants (n=412). Data are presented as mean±S.D. P values were determined by ordinary one-way ANOVA or student's t-test.

FIG. 13 shows a table presenting raw data showing gut bacteria species abundances and TyG level in human participants, Related to FIG. 7A.

FIG. 14 shows a table presenting raw data showing phospho-peptides analysis of tryptamine effects on insulin-related pathways (illustrated in FIG. 4), Related to FIG. 4A

FIG. 15 shows a table presenting the raw data showing BLASTP alignments with E-value<1e-5 and identity>30% to identify tryptamine producers in T2D subjects, Related to FIG. 6

FIG. 16 shows a table presenting the raw data showing correlation between fecal tryptamine with gut bacteria abundances in T2D subjects, Related to FIG. 6

FIG. 17 shows a table presenting the raw data showing MRM transition and parameters used in targeted metabolomics study.

### DETAILED DESCRIPTION

### Definition

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counterions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

As used herein, the term “selectively binds TAAR1” refers to a TAAR1 inhibitor to that preferably has an IC50 value for TAAR1 that is at least 2-fold lower than its IC50 value for other TAAR members (e.g., human TAAR2-6 or mouse TAAR2-16). In certain embodiments, the TAAR1 inhibitor has an IC50 value for TAAR1 that is at least 3-fold lower, at least 4-fold lower, at least 5-fold lower, at least 10-fold lower, at least 15-fold lower, at least 20-fold lower, at least 30-fold lower, at least 40-fold lower, at least 50-fold lower, at least 100-fold lower, at least 500-fold lower, or at least 1,000-fold lower than its IC50 value for other TAAR members.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “subject” as used herein, refers to an animal, typically a mammal or a human, that will be or has been the object of treatment, observation, and/or experiment. When the term is used in conjunction with administration of a compound described herein, then the subject has been the object of treatment, observation, and/or administration of the compound described herein.

The term “therapeutically effective amount” as used herein, means that amount of the compound or pharmaceutical agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

The present disclosure provides novel mechanistic insights into the contribution of gut microbes-derived tryptamine and phenethylamine to the induction of insulin resistance and the development of T2D. The present disclosure is in alignment with a recent clinical study which have identified an anaerobic, gram-positive bacterium namely Ruminococcus gnavus as a robust and independent predictor of metabolic syndrome in human. However, the causal effects of Ruminococcus gnavus and its related functional genes involved in metabolic syndrome remain unknown. Since the inventors' previous findings and recent studies have suggested Ruminococcus gnavus is a major tryptamine and phenethylamine producer by decarboxylating tryptophan and phenylalanine in human gut, they showed monoassociation of Ruminococcus gnavus led to insulin resistance and glucose intolerance concomitantly with elevated fecal tryptamine and phenethylamine levels in germ-free mice. In addition, it was shown that colonization of a gut microbe Lactobacillus casei engineered with Ruminococcus gnavus-derived decarboxylase (TDC) to produce tryptamine and phenethylamine led to insulin resistance in antibiotics-treated mice. It was found that fecal tryptamine and phenethylamine levels were both positively correlated with glucose intolerance in T2D subjects and negatively correlated with the improvement of glucose tolerance and insulin sensitivity in T2D subjects in a dietary fiber intervention study. They further demonstrated that gut microbes produce tryptamine and phenethylamine that in turn impair insulin signaling in the metabolic tissues via activation of the trace amine-associated receptor 1 (TAAR1)-extracellular signal-regulated kinase (ERK) signaling axis. Inhibition of TAAR1 alleviated impaired insulin sensitivity induced by the colonization of Ruminococcus gnavus in antibiotics-treated mice. These findings not only provide new insights into the causal role of gut dysbiosis in the pathogenesis of insulin resistance, but also construct fundamental knowledge for developing gut microbiota-based therapeutics for the management of metabolic syndrome.

Provided herein is a method of treating a metabolic disorder in a subject in need thereof, the method comprising: providing a fecal sample obtained from the subject; determining the amount of one or more markers selected from the group consisting of phenethylamine, tryptamine and Ruminococcus gnavus in the fecal sample; determining based on the amount of the one or more markers in the fecal sample that the subject has the metabolic disorder; and administering a therapeutically effective amount of a trace amine-associated receptor 1 (TAAR1) inhibitor to the subject.

The metabolic disorder can be insulin resistance, glucose intolerance, hyperglycemia, hyperlipidemia, pre-diabetes, type 2 diabetes, or a combination thereof. In certain embodiments, the metabolic disorder comprises insulin resistance.

The TAAR1 inhibitor can be a small molecule, antibody, protein, or the like. In certain embodiments, the TAAR1 inhibitor is a small molecule.

US2009/0036420A1, which is hereby incorporated by reference in its entirety, discloses the use of a TAAR1 inhibitor for treating a CNS disorder, wherein the TAAR1 inhibitor is a compound of Formula 1:

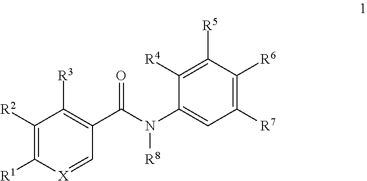

or a pharmaceutically acceptable salt thereof, wherein
n is 0, 1, 2 or 3;
p is 0 or 1;
R¹ is hydrogen, halogen, C₁-C₆ alkyl, C₁-C₆alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;
X is —C(R⁹)═ or —N═;
R⁹ is hydrogen, C₁-C₆ alkoxy, NO₂, or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

In certain embodiments, the TAAR1 inhibitor is selected from the group consisting of N-(3-methoxy-phenyl)-4-(4-methyl-piperidin-1-yl)-3-nitro-benzamide, N-(3-methoxy-phenyl)-3-nitro-4-propylamino-benzamide, 4-benzylamino-N-(3-methoxy-phenyl)-3-nitro-benzamide, 4-ethylamino-N-(3-methoxy-phenyl)-3-nitro-benzamide, 4-isopropylamino-N-(3-methoxy-phenyl)-3-nitro-benzamide, 4-azetidin-1-yl-N-(3-methoxy-phenyl)-3-nitro-benzamide, N-(3-methoxy-phenyl)-3-nitro-4-pyrrolidin-1-yl-benzamide, N-(3-methoxy-phenyl)-3-nitro-4-piperidin-1-yl-benzamide, N-(3-methoxy-phenyl)-3-nitro-4-phenylamino-benzamide, N-(3-methoxy-phenyl)-4-pyrrolidin-1-yl-3-trifluoromethyl-benzamide, 4-(2-methoxy-ethylamino)-N-(3-methoxy-phenyl)-3-trifluoromethyl-benzamide, 4-azetidin-1-yl-N-(3-methoxy-phenyl)-3-trifluoromethyl-benzamide, N-(3-methoxy-phenyl)-4-piperidin-1-yl-3-trifluoromethyl-benzamide, N-(3-methoxy-phenyl)-4-(4-methyl-piperidin-1-yl)-3-trifluoromethyl-benzamide, N-(3-methoxy-phenyl)-4-propylamino-3-trifluoromethyl-benzamide, 4-butylamino-N-(3-methoxy-phenyl)-3-trifluoromethyl-benzamide, 4-benzylamino-N-(3-methoxy-phenyl)-3-trifluoromethyl-benzamide, 4-ethylamino-N-(3-methoxy-phenyl)-3-trifluoromethyl-benzamide, 4-isopropylamino-N-(3-methoxy-phenyl)-3-trifluoromethyl-benzamide, N-(3-methoxy-phenyl)-4-morpholin-4-yl-3-trifluoromethyl-benzamide, N-(3-ethyl-phenyl)-4-pyrrolidin-1-yl-3-trifluoromethyl-benzamide, N-(3-ethoxy-phenyl)-4-pyrrolidin-1-yl-3-trifluoromethyl-benzamide, N-(3-isopropyl-phenyl)-4-pyrrolidin-1-yl-3-trifluoromethyl-benzamide, N-(3-isopropoxy-phenyl)-4-pyrrolidin-1-yl-3-trifluoromethyl-benzamide, N-(3-acetyl-phenyl)-4-pyrrolidin-1-yl-3-trifluoromethyl-benzamide, N-(3-fluoro-phenyl)-4-pyrrolidin-1-yl-3-trifluoromethyl-benzamide, N-(3-chloro-phenyl)-4-pyrrolidin-1-yl-3-trifluoromethyl-benzamide, N-(3-bromo-phenyl)-4-pyrrolidin-1-yl-3-trifluoromethyl-benzamide, 4-pyrrolidin-1-yl-N-m-tolyl-3-trifluoromethyl-benzamide, N-(3-difluoromethoxy-phenyl)-4-pyrrolidin-1-yl-3-trifluoromethyl-benzamide, 4-pyrrolidin-1-yl-N-[3-(1,1,2,2-tetrafluoro-ethoxy)-phenyl]-3-trifluoromethyl-benzamide, (rac,meso)-4-(3,5-dimethyl-piperidin-1-yl)-N-(3-methoxy-phenyl)-3-trifluoromethyl-benzamide, 4-azepan-1-yl-N-(3-methoxy-phenyl)-3-trifluoromethyl-benzamide, 4-(4-cyano-piperidin-1-yl)-N-(3-methoxy-phenyl)-3-trifluoromethyl-benzamide, N-(3-methoxy-phenyl)-3-trifluoromethyl-4-(4-trifluoromethyl-piperidin-1-yl)-benzamide, N-(3-chloro-phenyl)-6-piperazin-1-yl-nicotinamide, N-(3-chloro-phenyl)-6-(4-methyl-piperazin-1-yl)-nicotinamide, 5-chloro-N-(3-chloro-phenyl)-6-methylamino-nicotinamide, 5-chloro-N-(3-chloro-phenyl)-6-isopropylamino-nicotinamide, 5-chloro-N-(3-chloro-phenyl)-6-(2-methoxy-ethylamino)-nicotinamide, 5-chloro-N-(3-chloro-phenyl)-6-pyrrolidin-1-yl-nicotinamide, 3′-chloro-4-methyl-3,4,5,6-tetrahydro-2H-[1,2′]bipyridinyl-5′-carboxylic acid (3-chloro-phenyl)-amide, 5-chloro-N-(3-chloro-phenyl)-6-ethylamino-nicotinamide, 5-chloro-N-(3-chloro-phenyl)-6-propylamino-nicotinamide, 6-butylamino-5-chloro-N-(3-chloro-phenyl)-nicotinamide, 6-azetidin-1-yl-5-chloro-N-(3-chloro-phenyl)-nicotinamide, 3′-chloro-3,4,5,6-tetrahydro-2H-[1,2′]bipyridinyl-5′-carboxylic acid (3-chloro-phenyl)amide, 5-chloro-N-(3-chloro-phenyl)-6-(4-methyl-piperazin-1-yl)-nicotinamide, N-(3-methoxy-phenyl)-6-pyrrolidin-1-yl-5-trifluoromethyl-nicotinamide, 6-benzylamino-N-(3-methoxy-phenyl)-5-trifluoromethyl-nicotinamide, 6-isopropylamino-N-(3-methoxy-phenyl)-5-trifluoromethyl-nicotinamide, 4-methyl-3′-trifluoromethyl-3,4,5,6-tetrahydro-2H-[1,2′]bipyridinyl-5′-carboxylic acid (3-methoxy-phenyl)-amide, 5-chloro-N-(3-methoxy-phenyl)-6-pyrrolidin-1-yl-nicotinamide, 3′-chloro-4-methyl-3,4,5,6-tetrahydro-2H-[1,2′]bipyridinyl-5′-carboxylic acid (3-methoxy-phenyl)-amide, 6-butylamino-5-chloro-N-(3-methoxy-phenyl)-nicotinamide, 5-chloro-N-(3-chloro-phenyl)-6-piperazin-1-yl-nicotinamide, 4-chloro-N-phenyl-3-trifluoromethyl-benzamide, 4-chloro-N-(3-methoxy-phenyl)-3-nitro-benzamide, 4-bromo-N-(3-methoxy-phenyl)-3-nitro-benzamide, 3-chloro-4-fluoro-N-(3-methoxy-phenyl)-benzamide, 3-bromo-4-fluoro-N-(3-methoxy-phenyl)-benzamide, 4-fluoro-N-(3-methoxy-phenyl)-3-trifluoromethyl-benzamide, 4-fluoro-N-(3-methoxy-phenyl)-3-nitro-benzamide, 3,4-dichloro-N-[3-(2,5-dimethyl-imidazol-1-ylmethyl)-phenyl]-benzamide, 3,4-dichloro-N-phenyl-benzamide, 4-chloro-N-(3-methoxy-phenyl)-3-trifluoromethyl-benzamide, 3,4-dichloro-N-phenyl-benzamide, 3,3′,4-trichlorobenzanilide, 3,4-dichloro-N-(3-chloro-phenyl)-benzamide, 5,6-dichloro-N-(3-chloro-phenyl)-nicotinamide, 5,6-dichloro-N-(3-methoxy-phenyl)-nicotinamide, 6-chloro-N-(3-methoxy-phenyl)-5-trifluoromethyl-nicotinamide, N-(3-methoxy-phenyl)-3-nitro-4-propylamino-benzamide, 4-benzylamino-N-(3-methoxy-phenyl)-3-nitro-benzamide, 4-ethylamino-N-(3-methoxy-phenyl)-3-nitro-benzamide, 4-isopropylamino-N-(3-methoxy-phenyl)-3-nitro-benzamide, N-(3-methoxy-phenyl)-3-nitro-4-phenylamino-benzamide, 4-(2-methoxy-ethylamino)-N-(3-methoxy-phenyl)-3-trifluoromethyl-benzamide, N-(3-methoxy-phenyl)-4-propylamino-3-trifluoromethyl-benzamide, 4-butylamino-N-(3-methoxy-phenyl)-3-trifluoromethyl-benzamide, 4-benzylamino-N-(3-methoxy-phenyl)-3-trifluoromethyl-benzamide, 4-ethylamino-N-(3-methoxy-phenyl)-3-trifluoromethyl-benzamide, 4-isopropylamino-N-(3-methoxy-phenyl)-3-trifluoromethyl-benzamide, 6-benzylamino-N-(3-methoxy-phenyl)-5-trifluoromethyl-nicotinamide, 6-isopropylamino-N-(3-methoxy-phenyl)-5-trifluoromethyl-nicotinamide, 6-butylamino-5-chloro-N-(3-methoxy-phenyl)-nicotinamide, N-(3-methoxy-phenyl)-4-(4-methyl-piperidin-1-yl)-3-nitro-benzamide, 4-azetidin-1-yl-N-(3-methoxy-phenyl)-3-nitro-benzamide, N-(3-methoxy-phenyl)-3-nitro-4-pyrrolidin-1-yl-benzamide, N-(3-methoxy-phenyl)-3-nitro-4-piperidin-1-yl-benzamide, N-(3-methoxy-phenyl)-4-pyrrolidin-1-yl-3-trifluoromethyl-benzamide, 4-azetidin-1-yl-N-(3-methoxy-phenyl)-3-trifluoromethyl-benzamide, N-(3-methoxy-phenyl)-4-piperidin-1-yl-3-trifluoromethyl-benzamide, N-(3-methoxy-phenyl)-4-(4-methyl-piperidin-1-yl)-3-trifluoromethyl-benzamide, N-(3-methoxy-phenyl)-4-morpholin-4-yl-3-trifluoromethyl-benzamide, N-(3-ethyl-phenyl)-4-pyrrolidin-1-yl-3-trifluoromethyl-benzamide, N-(3-ethoxy-phenyl)-4-pyrrolidin-1-yl-3-trifluoromethyl-benzamide, N-(3-isopropyl-phenyl)-4-pyrrolidin-1-yl-3-trifluoromethyl-benzamide, N-(3-isopropoxy-phenyl)-4-pyrrolidin-1-yl-3-trifluoromethyl-benzamide, N-(3-acetyl-phenyl)-4-pyrrolidin-1-yl-3-trifluoromethyl-benzamide, N-(3-fluoro-phenyl)-4-pyrrolidin-1-yl-3-trifluoromethyl-benzamide, N-(3-chloro-phenyl)-4-pyrrolidin-1-yl-3-trifluoromethyl-benzamide, N-(3-bromo-phenyl)-4-pyrrolidin-1-yl-3-trifluoromethyl-benzamide, 4-pyrrolidin-1-yl-N-m-tolyl-3-trifluoromethyl-benzamide, N-(3-difluoromethoxy-phenyl)-4-pyrrolidin-1-yl-3-trifluoromethyl-benzamide, 4-pyrrolidin-1-yl-N-[3-(1,1,2,2-tetrafluoro-ethoxy)-phenyl]-3-trifluoromethyl-benzamide, (rac,meso)-4-(3,5-dimethyl-piperidin-1-yl)-N-(3-methoxy-phenyl)-3-trifluoromethyl-benzamide, 4-azepan-1-yl-N-(3-methoxy-phenyl)-3-trifluoromethyl-benzamide, 4-(4-cyano-piperidin-1-yl)-N-(3-methoxy-phenyl)-3-trifluoromethyl-benzamide, N-(3-methoxy-phenyl)-3-trifluoromethyl-4-(4-trifluoromethyl-piperidin-1-yl)-benzamide, 4-methyl-3′-trifluoromethyl-3,4,5,6-tetrahydro-2H-[1,2′]bipyridinyl-5′-carboxylic acid (3-methoxy-phenyl)-amide, 5-chloro-N-(3-methoxy-phenyl)-6-pyrrolidin-1-yl-nicotinamide, 3′-chloro-3,4,5,6-tetrahydro-2H-[1,2′]bipyridinyl-5′-carboxylic acid (3-methoxy-phenyl)-amide, 3′-chloro-4-methyl-3,4,5,6-tetrahydro-2H-[1,2′]bipyridinyl-5′-carboxylic acid (3-methoxy-phenyl)-amide, and 5-chloro-N-(3-chloro-phenyl)-6-piperazin-1-yl-nicotinamide; or the TAAR1 inhibitor is selected from the group consisting of

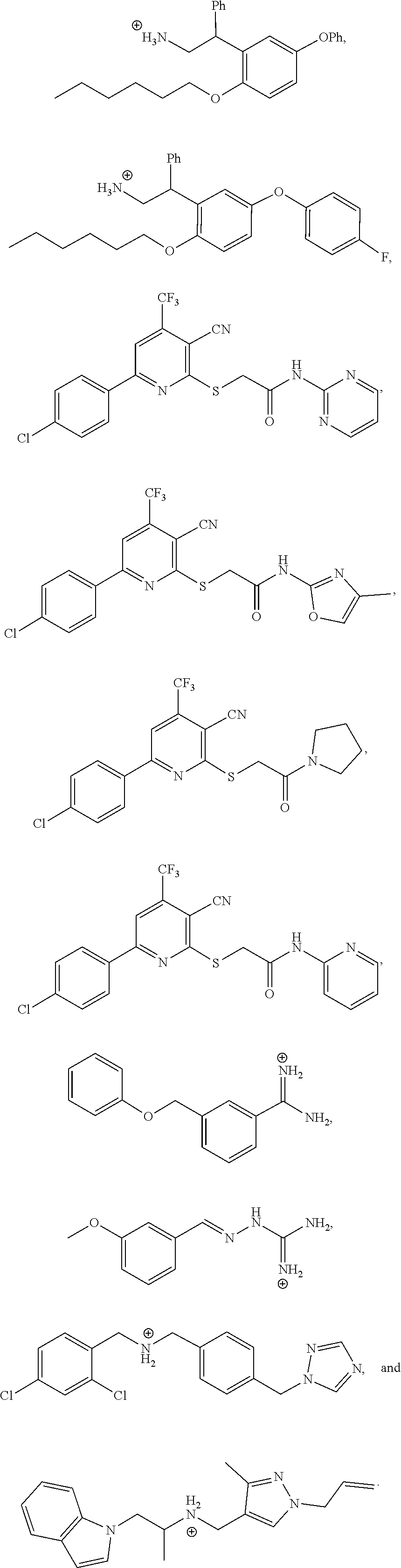

In certain embodiments, the TAAR1 inhibitor is N-(3-ethoxy-phenyl)-4-pyrrolidin-1-yl-3-trifluoromethyl-benzamide (EPPTB).

The subject typically refers to humans, but also to other animals, including, e.g., non-human primates, canines, equines, felines, ovines, porcines, rodents, and the like. In certain embodiments, the subject is a human.

In certain embodiments, a fecal sample obtained from the subject comprises a higher amount of one or more markers selected from the group consisting of Ruminococcus gnavus, phenethylamine, and tryptamine than an average of amount of the one or more markers in fecal samples obtained from healthy controls (e.g., subjects not suffering from the metabolic disorder).

The step of determining based on the amount of the one or more markers in the fecal sample that the subject has the metabolic disorder can comprise comparing the measured amount of the one or more markers in the fecal sample with the average amount of the one or more markers in fecal samples obtained from healthy controls (e.g., subjects not suffering from the metabolic disorder).

The one or more markers can be measured using any method known to those skilled in the art. In certain embodiments, the step of measuring the one or more markers can involve the use of one or more analytical tools, such as high performance liquid chromatography (LC), ultra-performance liquid chromatography (UPLC), liquid chromatography mass spectrometry (LCMS), liquid chromatography tandem mass spectrometry (LCMS/MS), microscopy, gram staining, examining the morphology of bacteria, nucleic acid analysis, e.g., using restriction enzymes, hybridization, polymerase chain reaction (PCR) amplification and/or sequencing, culture-based screening for nutrient requirements and/or antimicrobial sensitivity, analyzing fatty acid distribution, and/or test antigens, or combinations thereof.

The present disclosure also provides a method of diagnosing a metabolic disorder in a subject, the method comprising providing a fecal sample obtained from the subject; determining the amount of one or more markers selected from the group consisting of Ruminococcus gnavus, phenethylamine, and tryptamine in the fecal sample; determining based on the amount of the one or more markers in the fecal sample if the subject has the metabolic disorder.

In certain embodiments, the metabolic disorder is selected from the group consisting of insulin resistance, glucose intolerance, hyperglycemia, hyperlipidemia, pre-diabetes, type 2 diabetes, and combinations thereof. In certain embodiments, the metabolic disorder comprises insulin resistance.

In certain embodiments, the one or more markers comprises Ruminococcus gnavus.

The diagnostic method can further comprise the step of administering a therapeutically effective amount of a trace amine-associated receptor 1 (TAAR1) inhibitor to the subject. The TAAR1 inhibitor can be any TAAR1 inhibitor described herein. In certain embodiments, the TAAR1 inhibitor is N-(3-ethoxy-phenyl)-4-pyrrolidin-1-yl-3-trifluoromethyl-benzamide.

In certain embodiments, the TAAR1 inhibitor is a compound of Formula 1, the one or more markers comprises Ruminococcus gnavus and the metabolic disorder comprises insulin resistance

### Experiments

### Experimental Models and Subject Details

### Monkey Study

The first crab-eating macaques (Macaca fascicularis) metabolomics study is performed with ethics approval (No. YMB1704) by Yunnan Yinmore Biotechnology company (Kunming, China). The diagnosis of diabetes for monkeys was based on published criteria. Specifically, age-matched (10-23 years old) monkeys were categorized as normal (Define as FBG<75 mg/dL and Hb1Ac<3.5%), pre-diabetes (FBG 80-130 mg/dL and Hb1Ac 4.0%-6.0%) and diabetes (FBG>130 mg/dL and Hb1Ac>6.0%) (n=26 for each group). Biological samples including serum and feces were collected and stored at −80° C. until analysis. The second crab-eating macaques (n=5) intervention study is performed with ethics approval (No. HZ2021047) by Huazhen Biosciences company (Guangzhou, China). Biochemical parameters including OGTT and serum insulin level were measured before and after tryptamine treatment. No crab-eating macaques were treated with anti-diabetic medication during these studies.

### Mouse Study

All mouse studies were approved by the Committee on the Use of Human & Animal Subjects in Teaching & Research at Hong Kong Baptist University (Hong Kong SAR, China) and performed following the Animals (Control of Experiments) Ordinance of the Department of Health, Hong Kong SAR, China and reported following the ARRIVE guidelines. Male C57BL/6J mice aged 6-8 weeks and weighing 20-25 g were purchased from the Laboratory Animal Services Centre, The Chinese University of Hong Kong (Hong Kong SAR, China). The mice were housed with a 12-hour light/dark cycle at a controlled temperature of around 25° C. with free access to food and water.

Antibiotics-treated mice were generated using antibiotics cocktails containing 50 mg/kg vancomycin, 100 mg/kg neomycin, 100 mg/kg metronidazole, 100 mg/kg ampicillin, 50 mg/kg streptomycin via oral gavage for 9 days (one time per day) as previously reported. Germ-free mice were purchased from Nanjing GemPharmatech Co. and the monoassociation study in germ-free mice was performed in sterile plastic isolators as previously reported.

Taar1 (NM_053205) knockout mice (C57BL/6J) were generated using CRISPR/Cas-mediated genome engineering and provided by Cyagen Biosciences (Suzhou) Inc. The Taar1 knockout mice and their wildtype littermates were genotyped using PCR and southern blot by following primers [F1: GACAAAACGTAGTTGGAAGACTGA (SEQ ID NO:1), R1: GTGTGCCTAGAAACCTTAACATCTG (SEQ ID NO:2), R2: AATGTTTGTGATAGCGTGGCAAAG (SEQ ID NO:3)].

### Human Study

The first cohort of healthy controls and T2D subjects was approved by the Research Ethics Committee of Shanghai Jiao Tong University Affiliated Sixth People's Hospital. Written informed consent was signed and obtained from all participants. Subjects with fasting blood glucose<6.1 mmol/L were classified as healthy controls (HC), whereas those with fasting blood glucose>7.0 mmol/L or OGTT (2h)>11.1 mmol/L were classified as T2D.

The second cohort GUT2D study was approved by the Ethics Committee at the School of Life Sciences and Biotechnology, Shanghai Jiao Tong University (Ref ID: 2014-016). Written informed consent was obtained from all participants. The trial was registered in the Chinese Clinical Trial Registry (ChiCTR-TRC-14004959). Participants with T2D received either acarbose plus the usual diet (control; U group) or acarbose plus the WTP diet (intervention; W group) for 84 days.

The third cohort study of healthy volunteers was approved by the Ethics Committee of Hong Kong Baptist University (Ref ID: HASC/15-16/0300 and HASC/16-17/0027). Written informed consent was signed and obtained from all participants. Subjects with morbid obesity or diabetes or with fasting blood glucose>7.0 mmol/L were excluded from this study.

### Cell Study

3T3-L1 adipocytes (ATCC CL-173) were cultured and maintained in DMEM with 10% (v/v) FBS. For the glucose uptake assay, 3T3-L1 cells were serum- and glucose-starved for 3 hours and then incubated with glucose, FBS, insulin and tryptamine for 30 min as indicated. To assess the effect of tryptamine on insulin signaling, 3T3-L1 cells were pre-treated with or without tryptamine, ERK inhibitor or TAAR1 antagonist EPPTB, and then treated with insulin at the indicated concentration. The effect of IAA on insulin signaling was evaluated by treating 3T3-L1 cells with or without IAA followed by insulin for the indicated times. Tryptamine, U0126 and EPPTB were dissolved in DMSO at 100 mM as a stock solution. For cAMP measurements, 3T3-L1 cells were pre-treated with or without EPPTB for 60 min and then treated with tryptamine for the indicated times.

### Bacterial Strains Culture

Tryptamine-producing Lactobacillus casei was constructed employing the tryptophan decarboxylase (TDC) gene from Ruminococcus gnavus. The TDC gene was cloned into the vector and the resulting plasmid was transferred into L. casei as previously described. Successful insertion of the TDC gene into the L. casei was confirmed by PCR and the production of tryptamine when cultured in an MRS broth containing 0.25% tryptophan. The engineered L. casei TDC+ and vector-only L. casei were grown on MRS agar plates containing erythromycin (50 μg/mL) and further grown in MRS broth. The L. casei TDC+ and vector-only For administration, L. casei were collected from the medium by centrifugation at 3,000 rpm for 10 min at room temperature. L. casei inoculums were prepared in 300 μL of sterilized PBS and then administered to antibiotics-treated mice by oral gavage.

Primers used for determining TDC plasmid are [F1: CGGTCCTCGGGATATGATAAGA (SEQ ID NO:4); R1: GACCCTCCGCTTACAAAGAC (SEQ ID NO:5)].

Primers used for Sanger sequencing are [F1: CGGTCCTCGGGATATGATAAGA (SEQ ID NO:4); R1: GACCCTCCGCTTACAAAGAC (SEQ ID NO:5); R2: AGGCAGCTGATCTCAACAATG (SEQ ID NO:6)].

### Study Methods Details

### Fecal Suspension Administration

About 10 g of fecal samples were mixed with 5× sterilized 1x phosphate-buffered saline (PBS, m/v) and homogenized as fecal suspension. HFD-fed mice were orally administered with the fecal suspension derived from normal and diabetic monkeys at 4 g/kg daily for 5 days. On day 5, following a 12-h fast, an OGTT was performed to examine the effects of the fecal suspension from monkeys on glucose tolerance.

### Metabolomics Study

About 150 mg of feces were extracted with a 20× volume of 70% methanol (m/v) and then homogenized with steel beads. The samples were then centrifuged at 14,000 rpm at 4° C. for 15 min. About 200 μL of the supernatant was transferred to new tubes for LC-MS analysis. A pooled quality control (QC) sample was prepared by mixing equal amounts of each sample. An Agilent ultra-performance liquid chromatography (UPLC) system coupled to a tandem quadrupole-time-of-flight (Q-TOF) equipped with an AJS electrospray interface (G6540A) mass spectrometry was used for untargeted metabolomics as previously described.

An Agilent UPLC system coupled to a triple quadrupole (QQQ) 6460 mass spectrometry was used for targeted metabolomics. A Waters BEH 2.1×100 mm C18 1.7 μm column with a pre-column was used. The mobile phase used in LC-MS-QQQ was A: water with 0.1% formic acid and B: acetonitrile with 0.1% formic acid. The gradients were set as 2% B (0-0.5 min), 2-30% B (0.5-4 min), 30-100% B (4-6 min), 100% B (6-8 min), 100-2% B (8-8.1 min) and maintained in 2% B (8.1-10 min). The standards list, MRM transition and retention time were provided in (FIG. 17).

### Batch Culture of Fecal Samples

About 50 mg of fecal samples were mixed with 20× volume of sterilized 1× PBS (m/v) and homogenized with steel beads. The fecal suspension (20 μL) was inoculated in 2 mL Tryptic Soy Broth (TSB) supplemented with 0.25% tryptophan and incubated overnight under anaerobic conditions at 37° C. After incubation, 100 μL of the medium was then used and processed for quantification of tryptamine by LC-MS analysis following the serum protocol of LC-MS.

### Glucose and Insulin Tolerance Test

For the oral glucose tolerance test (OGTT), mice were fasted for 12 hours (overnight) and administered with tryptamine, ERK inhibitors, TAAR1 antagonist or IAA at indicated dosages. After 30 min, mice were given glucose at a dosage of 2 g/kg. Blood samples were collected from the tail vein for glucose measurement using Accu-Chek glucose meters at 0, 15, 30, 60, 90 and 120 min after the glucose challenge. For the insulin tolerance test (ITT), mice were fasted for 4 hours and administered with tryptamine, ERK inhibitors or a TAAR1 antagonist at indicated dosages. After 30 min, insulin (1 U/kg) was injected intraperitoneally into the mice. Blood glucose levels were measured as per OGTT. For the measurement of serum insulin and TG, serum was collected 120 min after the mice were treated with tryptamine.

### Tissue Distribution of Tryptamine

Tryptamine at a dosage of 5 mg/kg (dissolved in 0.5% CMC-Na) was orally administered to mice. After 15 min, mice were euthanized by isoflurane and sacrificed by cervical dislocation. Serum, liver, skeletal muscle and WAT were collected and stored at −80° C. until analysis. About 80 mg of biological tissues were extracted with a 20× volume of 70% methanol (m/v) and then homogenized with steel beads. The samples were then centrifuged at 14,000 rpm at 4° C. for 15 min. About 200 μL of the supernatant was transferred to new tubes for LC-MS analysis.

### Phospho-Proteomics Study

Mice were fasted for 4 hours and orally administered with tryptamine or vehicle. The WAT tissues were collected for the phosphoproteomics study. Mice tissue samples used for the TMT-labeled phosphoproteomics study were prepared as previously reported. An Easy nLC system (Thermo Fisher Scientific) with an Acclaim PepMap RSLC column (50 μm×15 cm) was used to separate the TMT-labelled peptides. The mobile phase used in LC-MS-Orbitrap was A: water with 0.1% formic acid and B: 80% acetonitrile, 20% water with 0.1% formic acid. The elution gradient was set as 0-5 min in 0-6% buffer B, 5-45 min in 6-28% buffer B, 45-50 min in 28%-38% buffer B, 50-55 min 38-100% buffer B and maintained during 50-60 min in 100% buffer B. The obtained MS/MS spectra were processed by Proteome Discoverer (Thermo Fisher Scientific) and searched using MASCOT engine 2.6. All protein sequences were aligned to the Mus musculus database downloaded from UniProt (http://www.uniprot.org). The proteins with a fold change>1.2 or <0.8 and a p-value<0.05 were considered as differentially expressed proteins. KEGG pathway annotation was performed using KOALA (KEGG Orthology And Links Annotation) to identify the significantly enriched pathways.

### Protein Analysis

Frozen tissues and harvested cells were lysed in RIPA buffer with a protease inhibitor cocktail. For western blotting, the cell lysates and tissue lysates were centrifuged at 15,000 rpm for 15 min at 4° C. The supernatant was mixed with 5× loading buffer and heated at 98° C. on a dry bath for 10 min. The target proteins were then detected in the samples as per manufacturer instructions. The blots were incubated with HRP-linked anti-rabbit IgG or anti-mouse IgG and reacted with enhanced chemiluminescence. The quantification of protein bands from western blots was analyzed using Image J.

### Characterization of Tryptamine-Producing Bacteria

The two reference tryptophan decarboxylase sequences of Ruminococcus gnavus (strain ATCC 29149/VPI C₇-9) and Clostridium sporogenes (strain ATCC 15579) were downloaded from ENA (A7B1V0 and J7SZ64). The identity between these two reference sequences was 26% based on BLASTP. To identify potential tryptophan decarboxylase sequences in the GUT2D dataset, BLASTP was used to align the two reference sequences against the nonredundant microbiome gene catalog constructed in the GUT2D study. The alignments were filtered with E-value<1e-5 and identity>30%. Repeated measures correlation coefficients between fecal metabolites and abundances of the 5 co-abundance groups were calculated as previously described.

### Quantification and Statistical Analysis

Data are expressed as average and SD or SEM values of at least triplicates. P-values were calculated using GraphPad Prism 8 andp-values less than 0.05 are considered statistically significant. Wilcoxon rank-sum test (one-tailed or two-tailed test) was employed to determine the differences in metabolomics data between subjects with and without T2D. Unpaired Student's t-tests or one-way ANOVA were employed in other settings as indicated.

### Results

Colonization with R. gnavus Impaired Insulin Sensitivity Accompanied by Tryptamine and Phenethylamine Overproduction

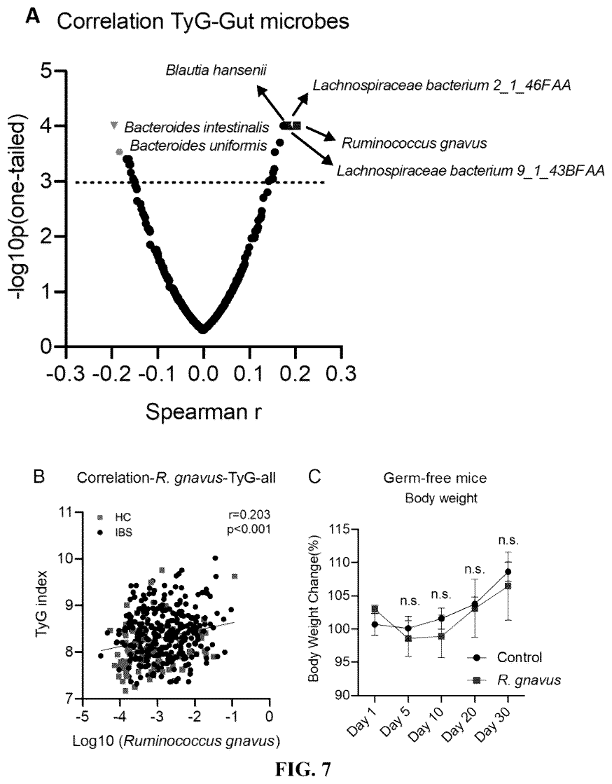

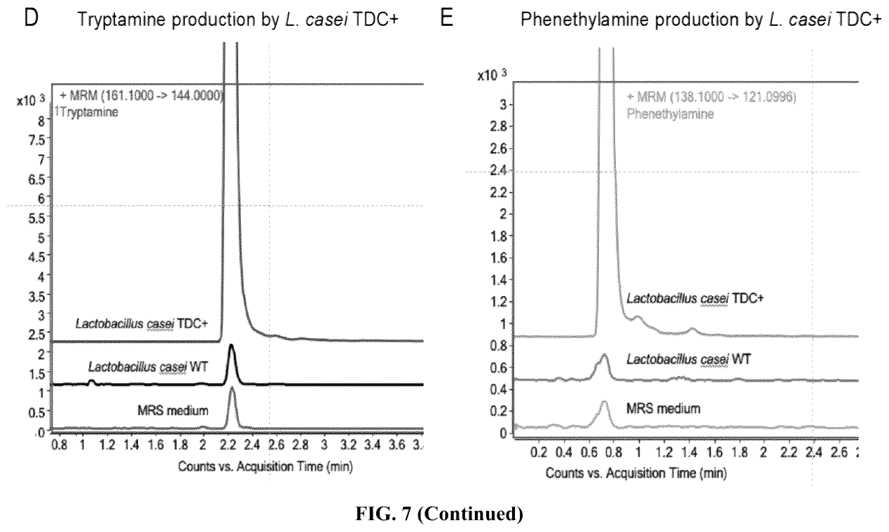

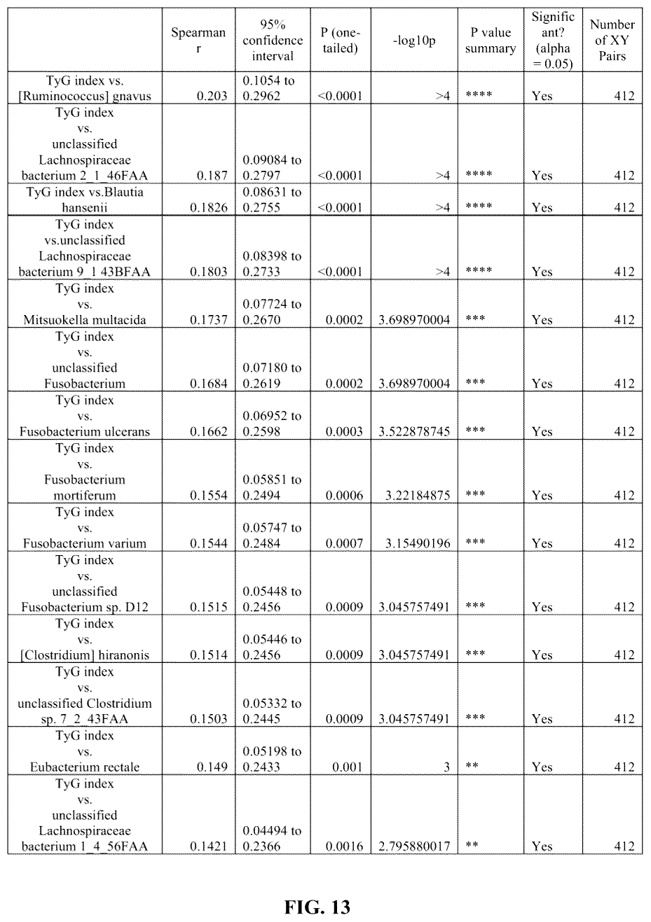

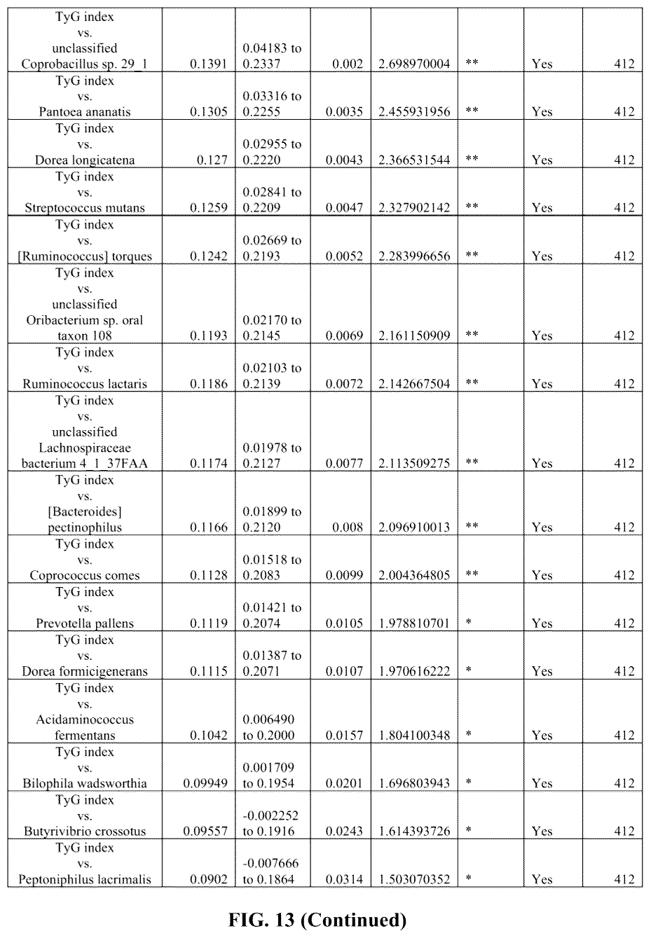

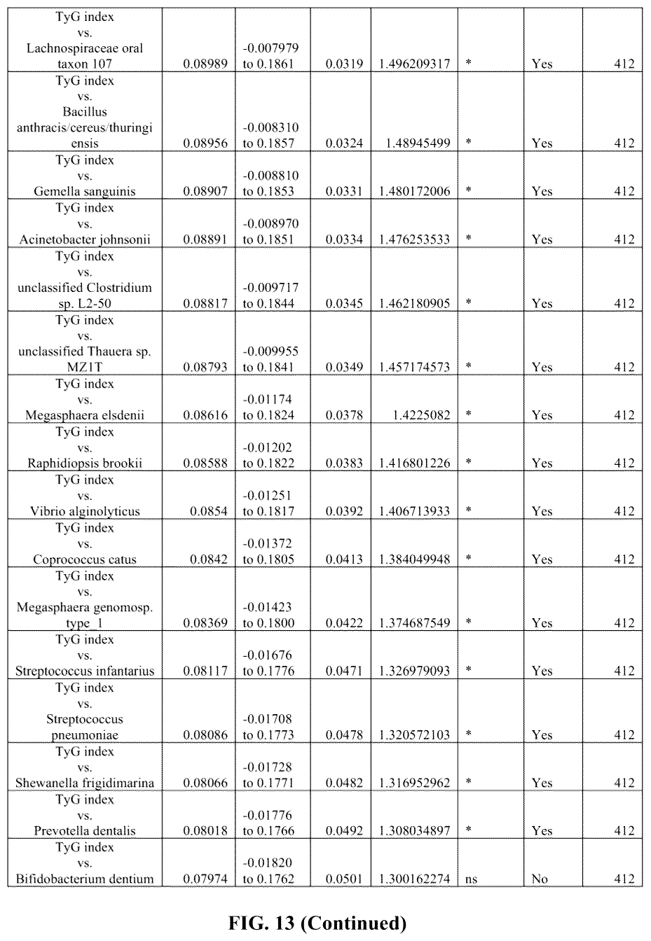

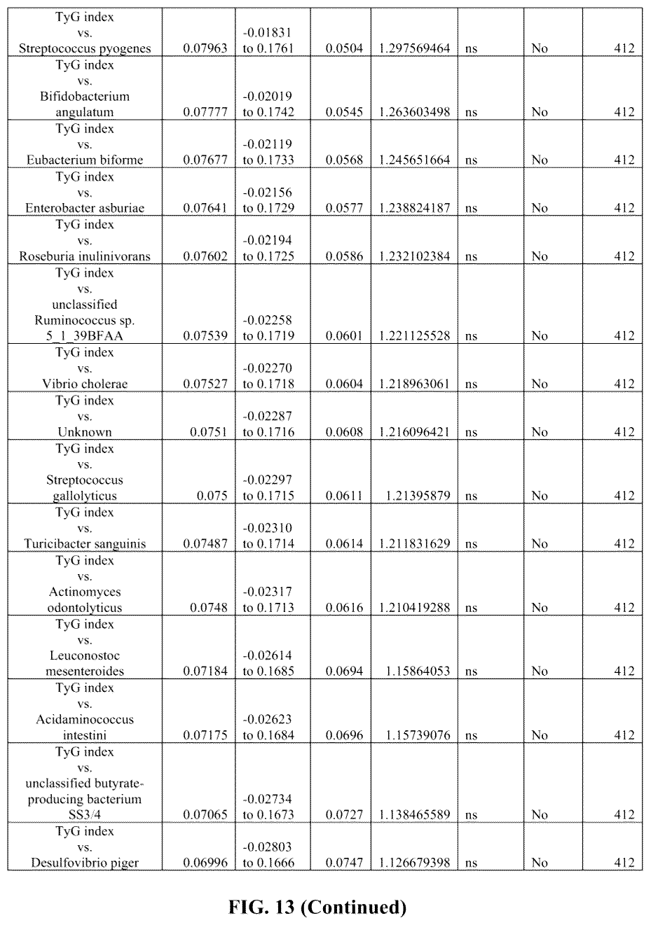

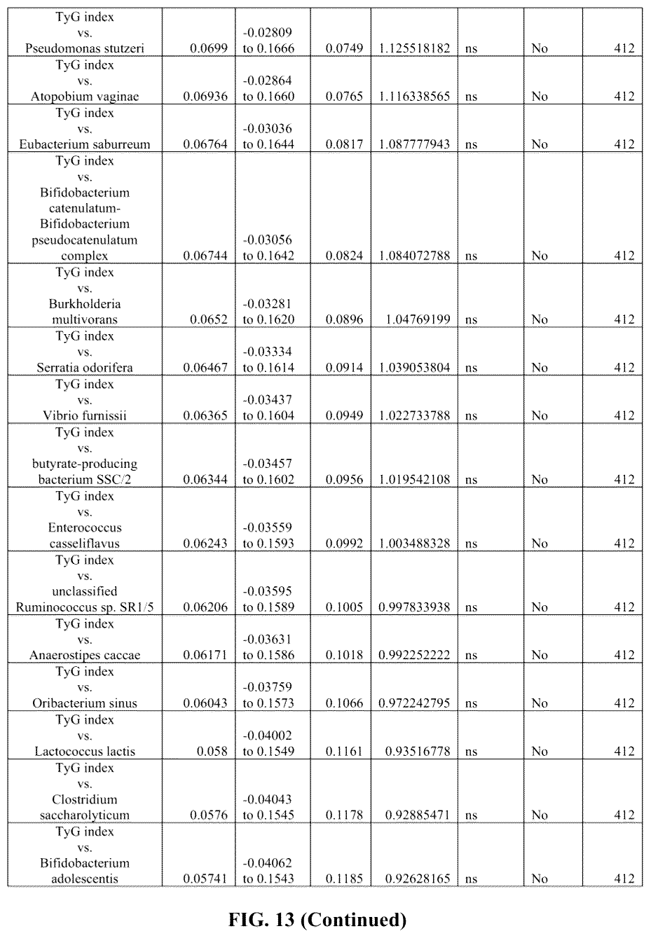

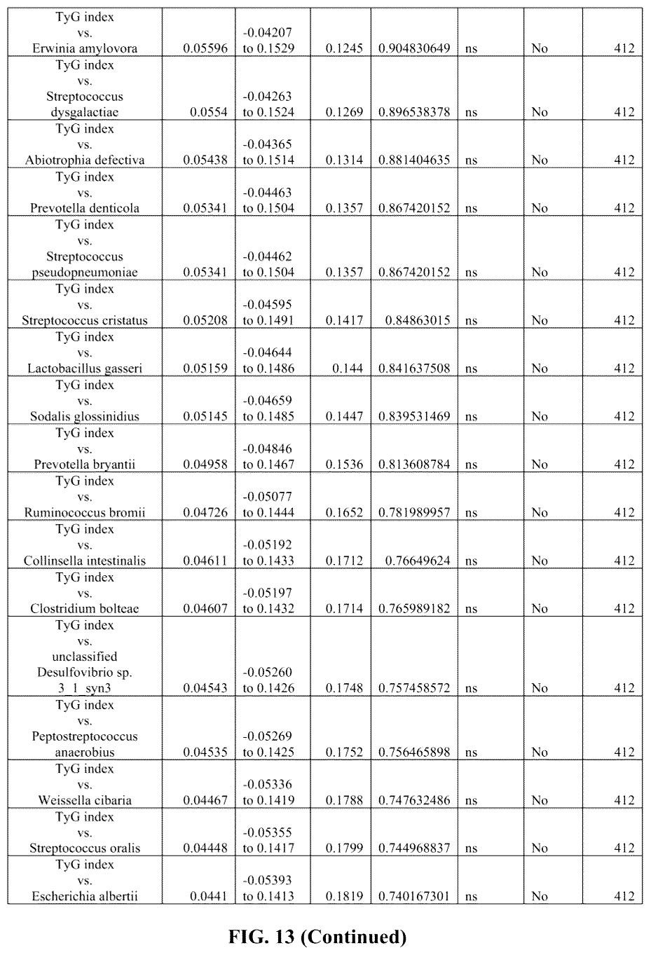

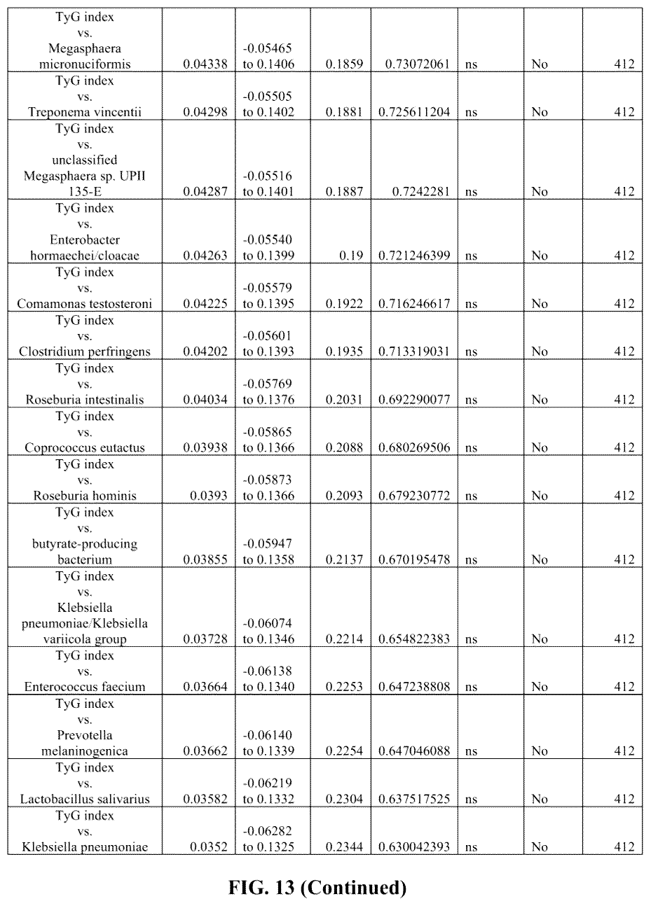

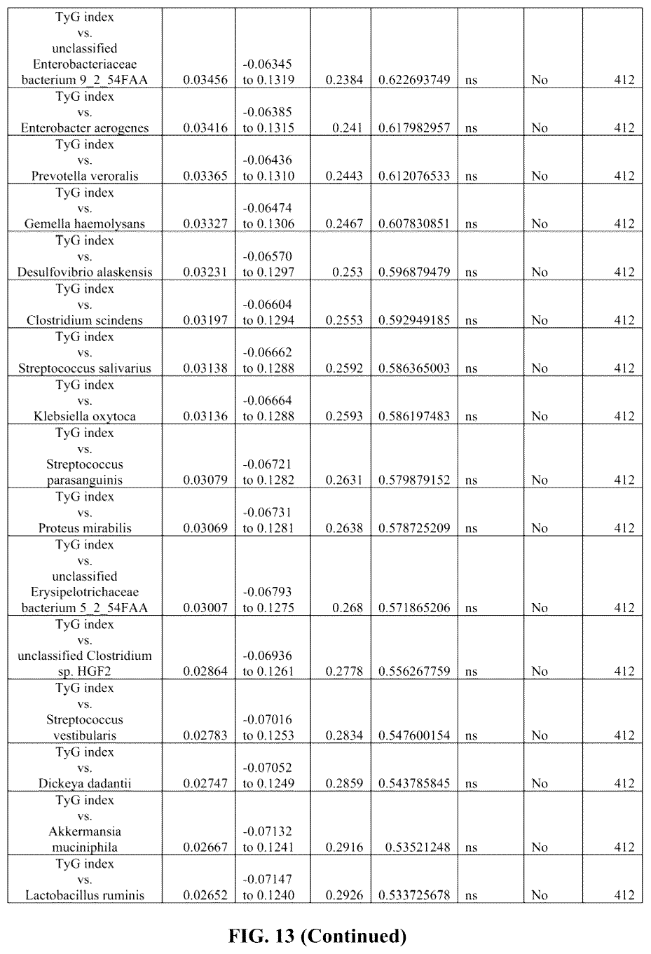

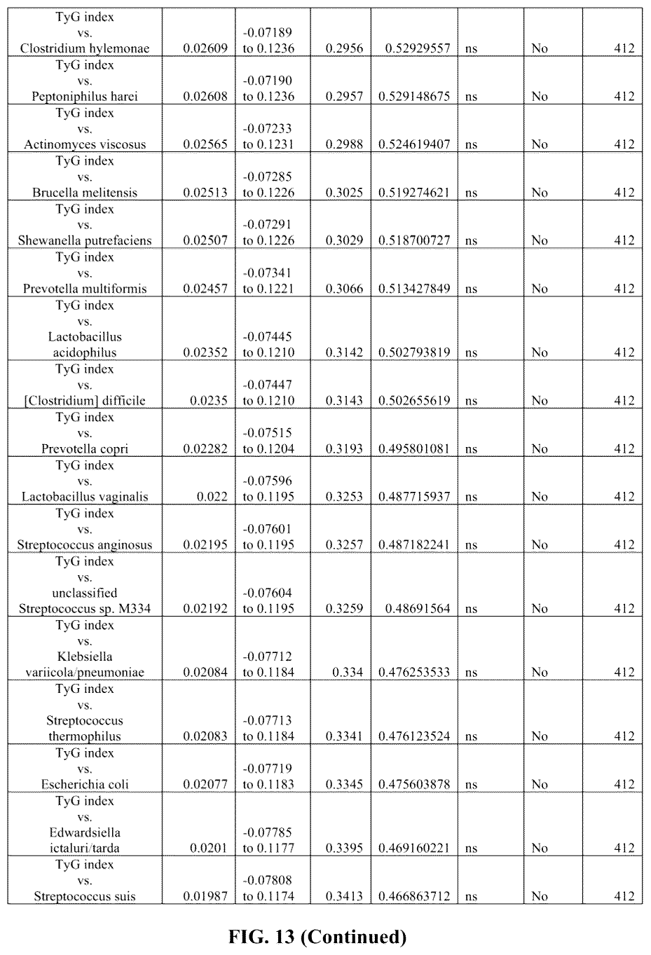

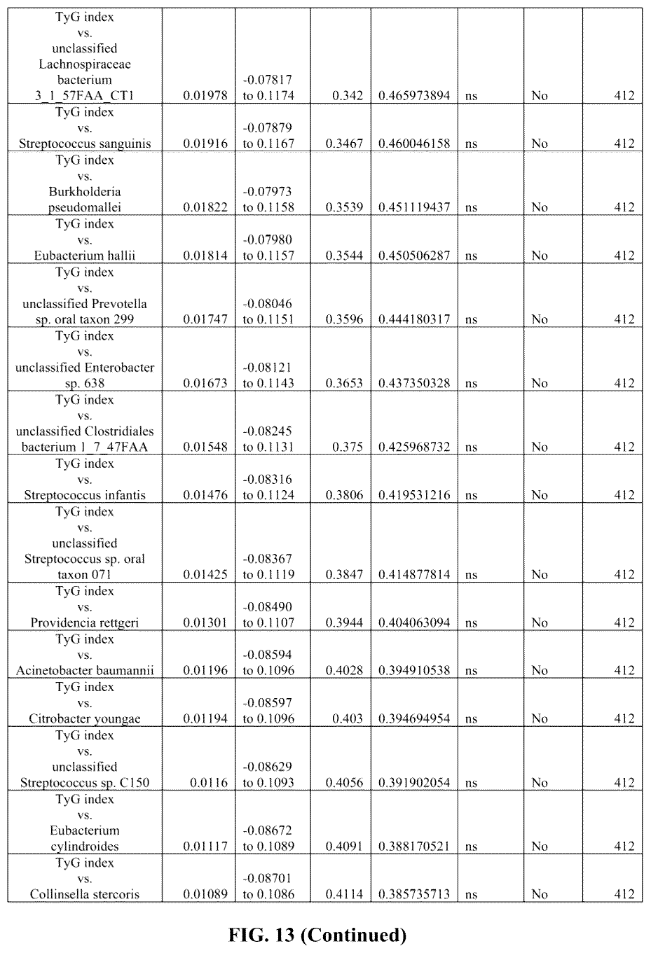

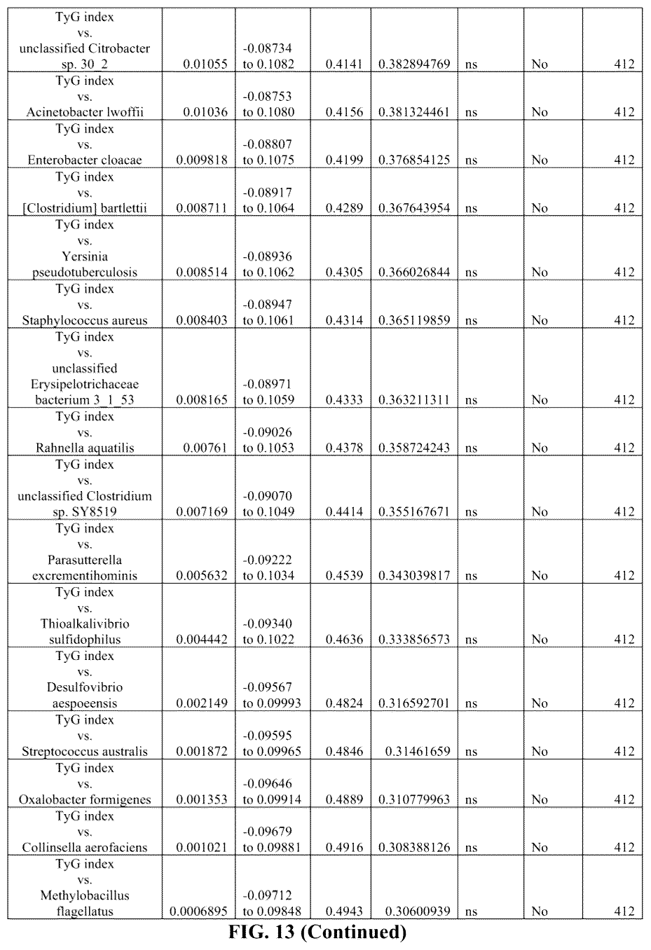

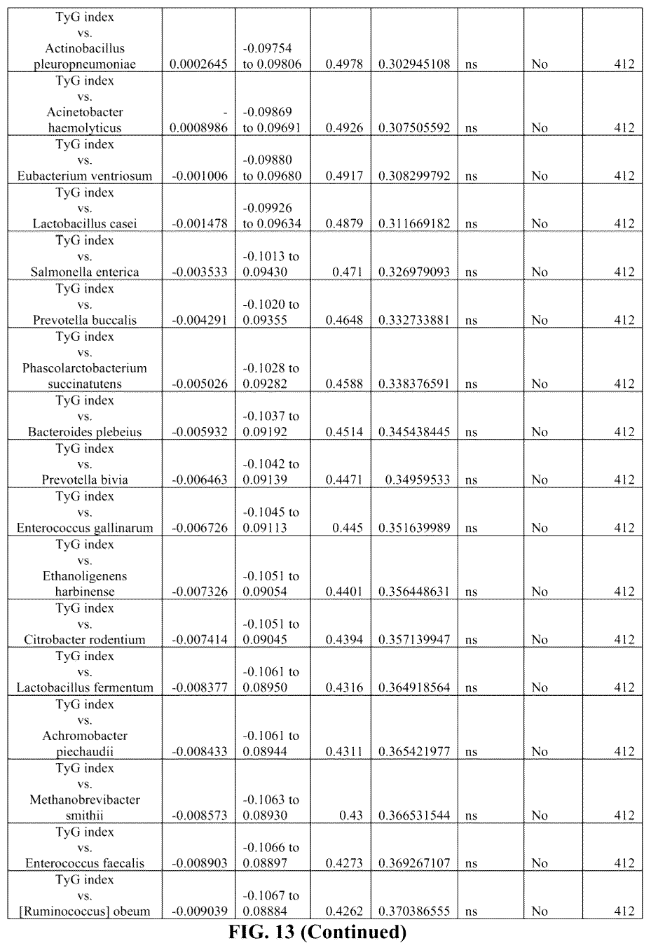

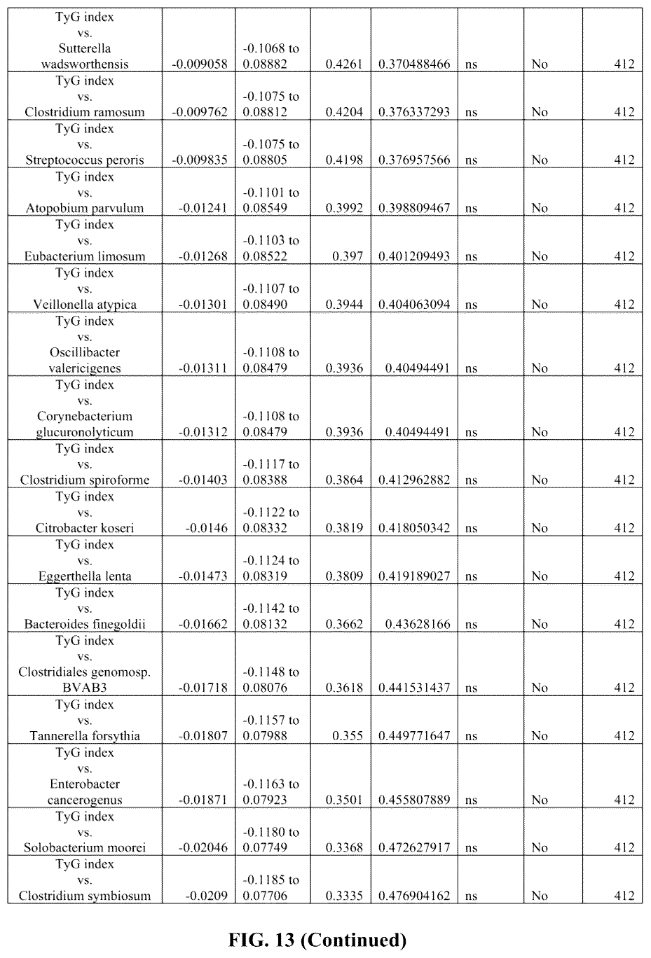

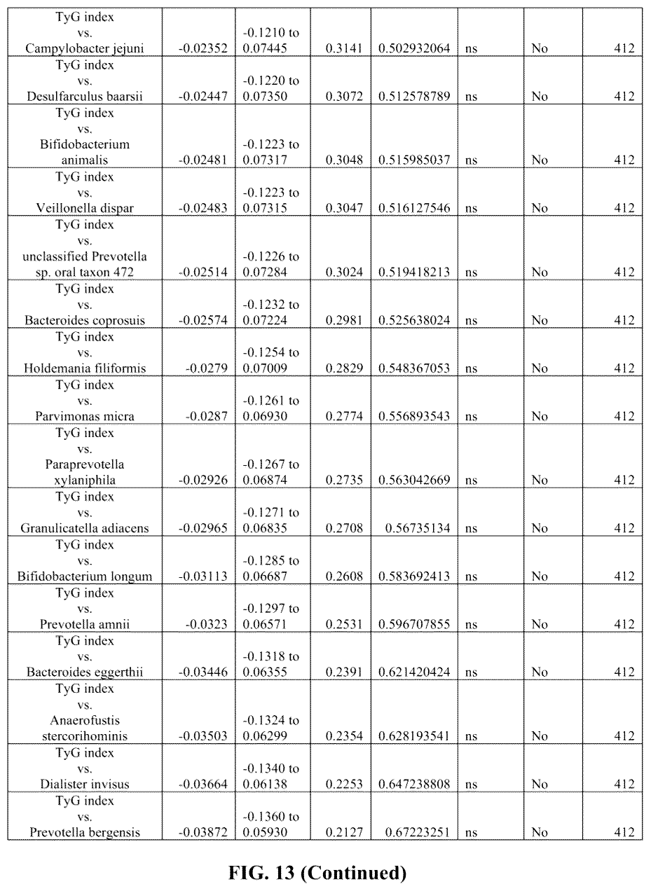

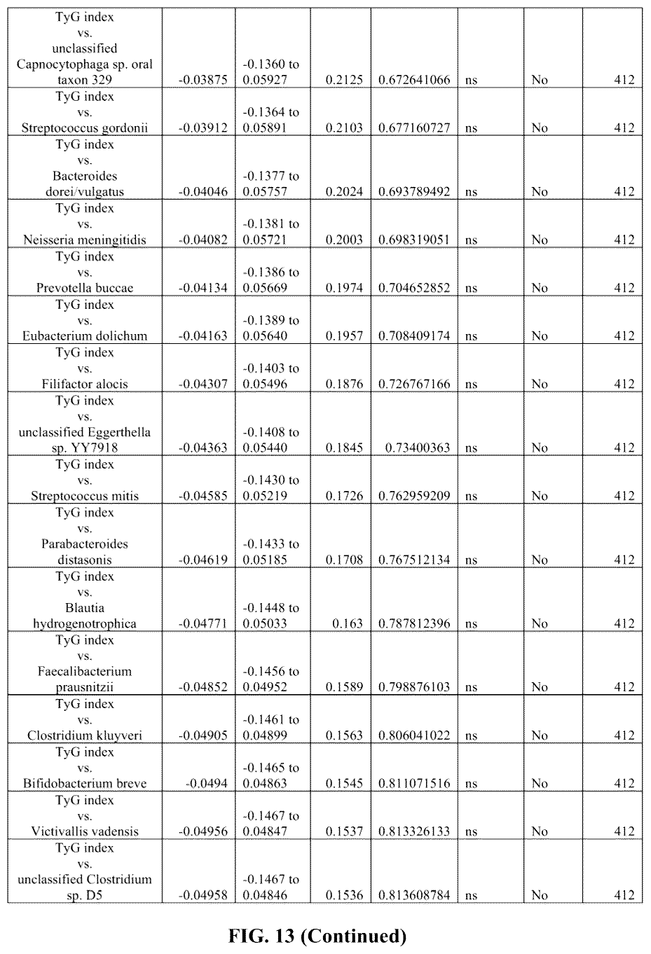

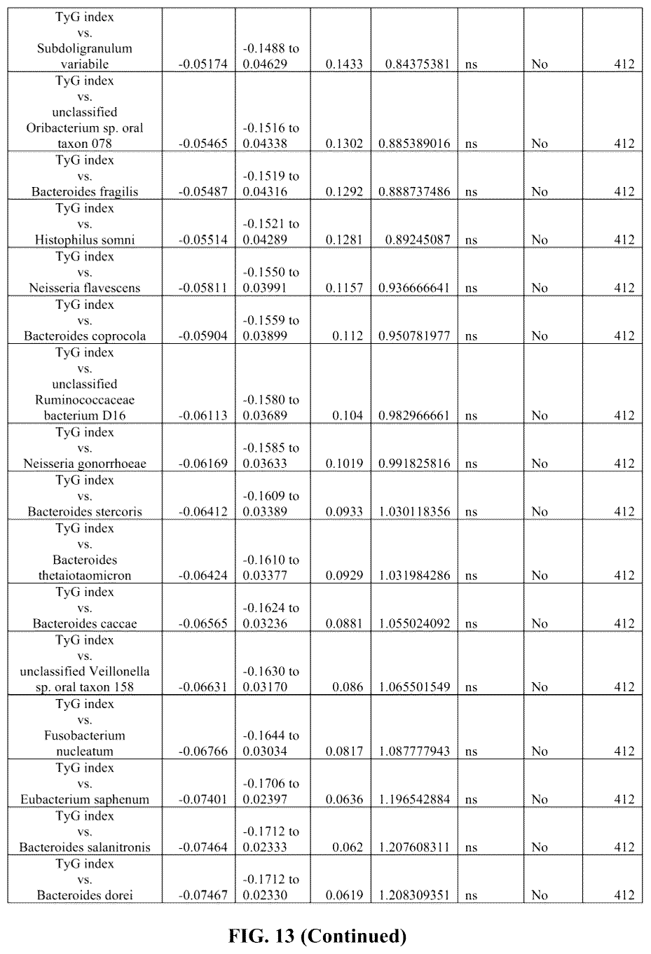

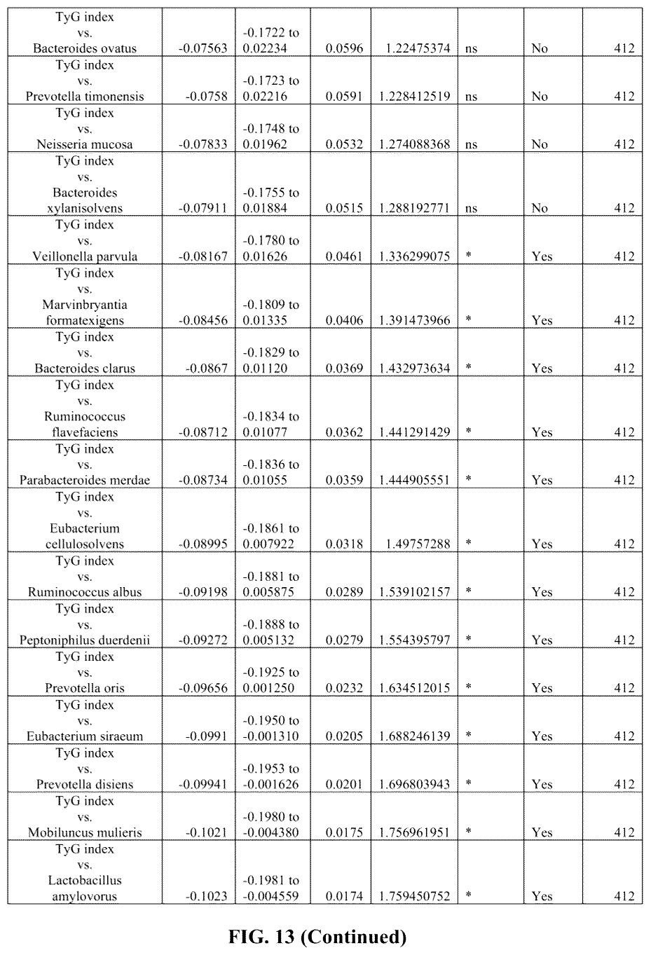

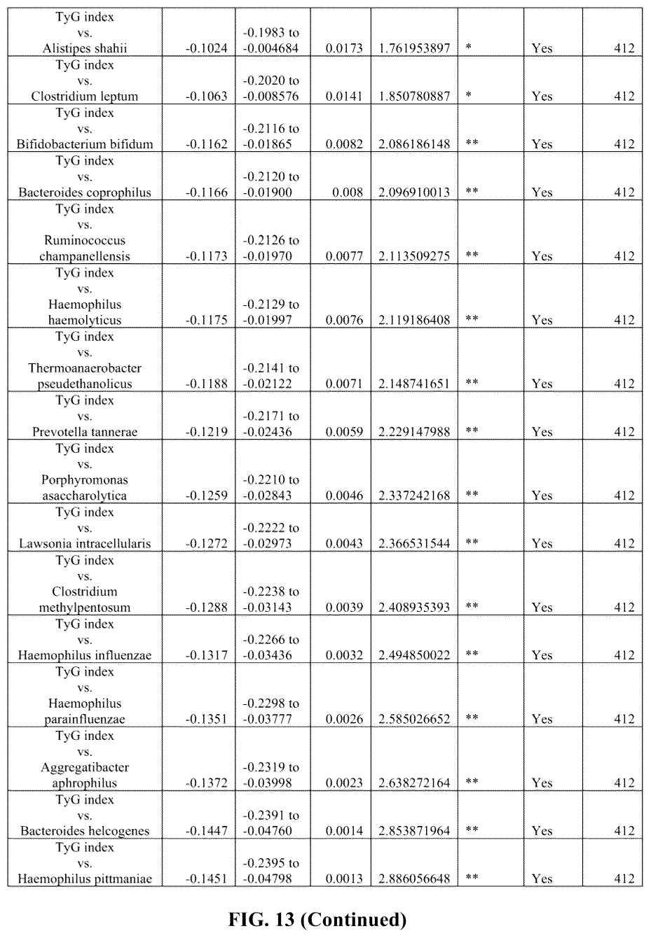

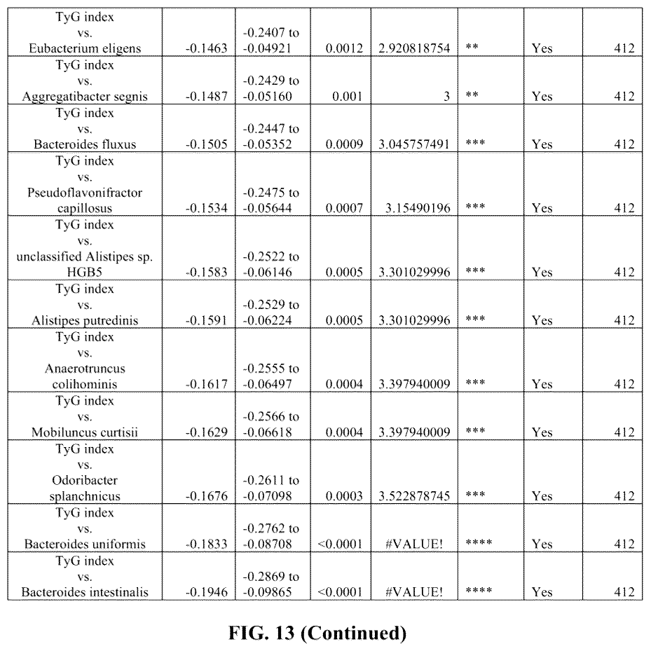

The inventors first analyzed the association between gut microbes with insulin resistance in the participants using their shotgun metagenomic sequencing data and triglyceride-glucose index (TyG), a marker of insulin resistance (FIG. 7A and FIG. 13). Among characterized gut microbes, R. gnavus is found positively correlated with TyG (r=0.203, p<0.001, FIG. 7B), which in alignment with a recent clinical study showing R. gnavus is associated with several features of metabolic syndrome including serum triglyceride (TG) and hemoglobin A1c (HbA1c).

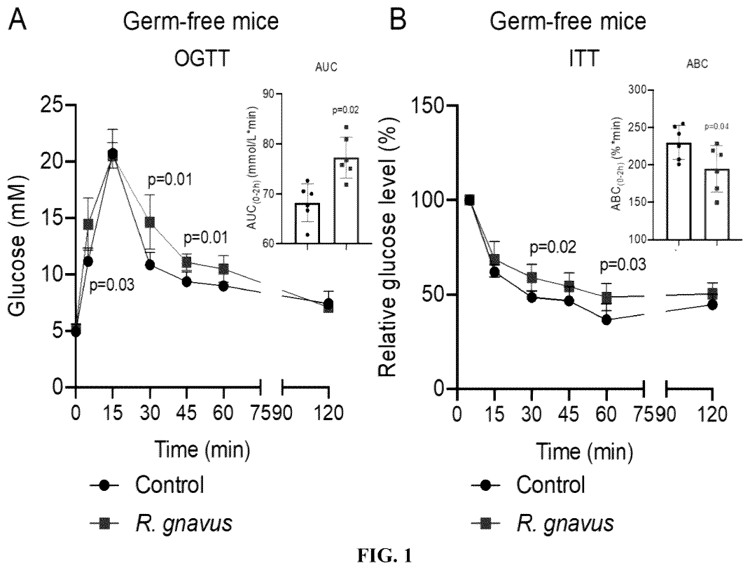

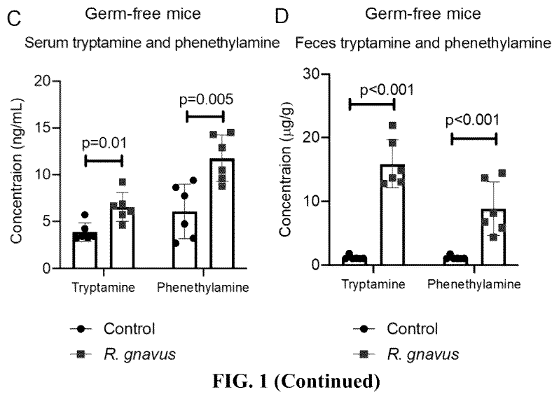

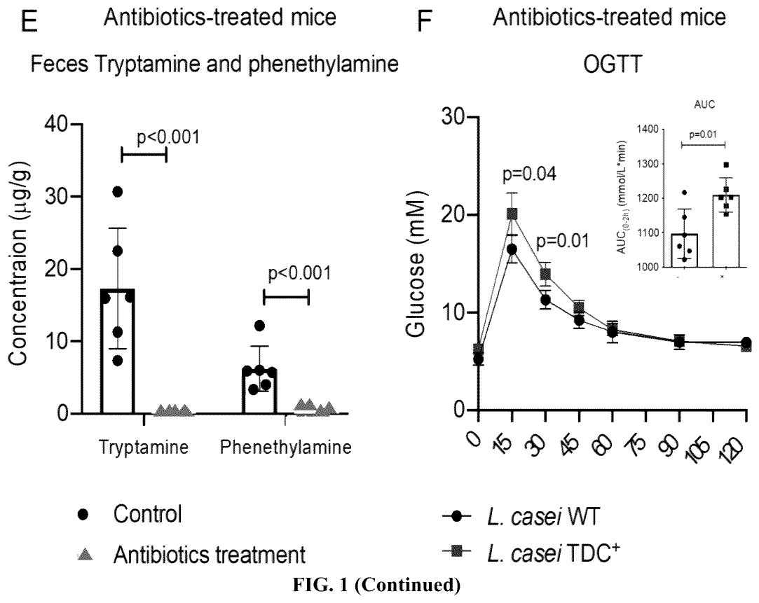

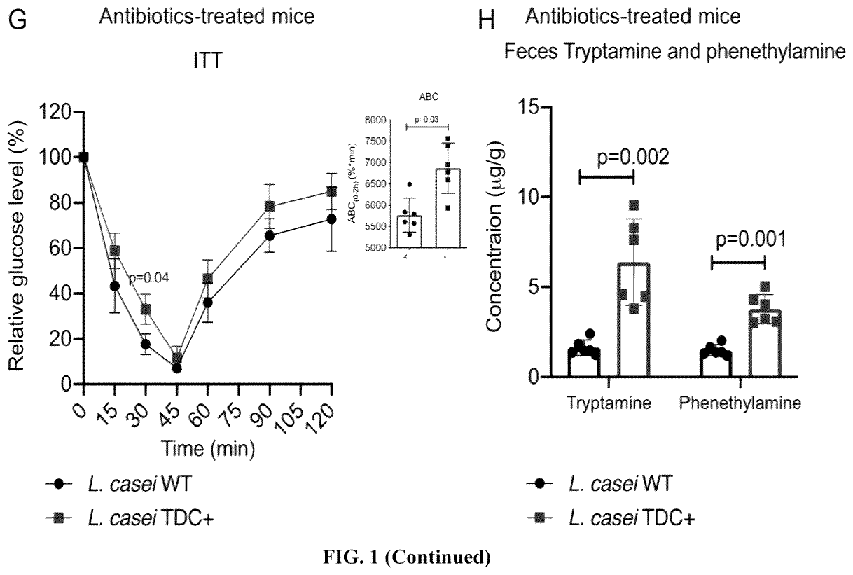

To evaluate the pathogenic role of R. gnavus in metabolic syndrome, the inventors colonized a human gut bacterium strain R. gnavus (ATCC 29149) in germ-free mice. Despite no significant changes in body weight (FIG. 7C), germ-free mice colonized with R. gnavus exhibited impaired glucose tolerance and reduced insulin sensitivity as determined by oral glucose tolerance test (OGTT) and insulin tolerance test (ITT) (p<0.05 in all cases, FIG. 1A-B).

As a primary gut microbe that is capable of decarboxylating tryptophan and phenylalanine into tryptamine and phenethylamine, elevated fecal tryptamine and phenethylamine levels were found along with impaired glucose tolerance and insulin sensitivity in germ-free mice colonized with R. gnavus (p<0.01 in all cases, FIG. 1C-D), revealing R. gnavus-derived decarboxylase (TDC) transforms dietary amino acids tryptophan and phenylalanine into tryptamine and phenethylamine in germ-free mice.

To further investigate whether tryptamine and phenethylamine produced by R. gnavus-derived TDC impair insulin sensitivity in vivo, the inventors ectopically expressed TDC gene from R. gnavus (ATCC 29149) in a commensal bacterium Lactobacillus casei, which does not produce tryptamine or phenethylamine. The inventors showed that both tryptamine and phenethylamine levels were significantly increased in the culture medium from the engineered L. casei TDC+strain when compared to the L. casei with empty vector and medium control after overnight inoculation (FIG. 7D-E). Colonization of L. casei TDC+strain induced higher OGTT and ITT indexes together with elevated fecal tryptamine and phenethylamine levels in antibiotics-treated mice compared with blank vector L. casei (p<0.05 in all cases, FIG. 1E-H), indicating that tryptamine and phenethylamine produced from R. gnavus-derived TDC may impair insulin sensitivity in vivo.

### The Positive Association Between Tryptamine/Phenethylamine and Glucose Intolerance

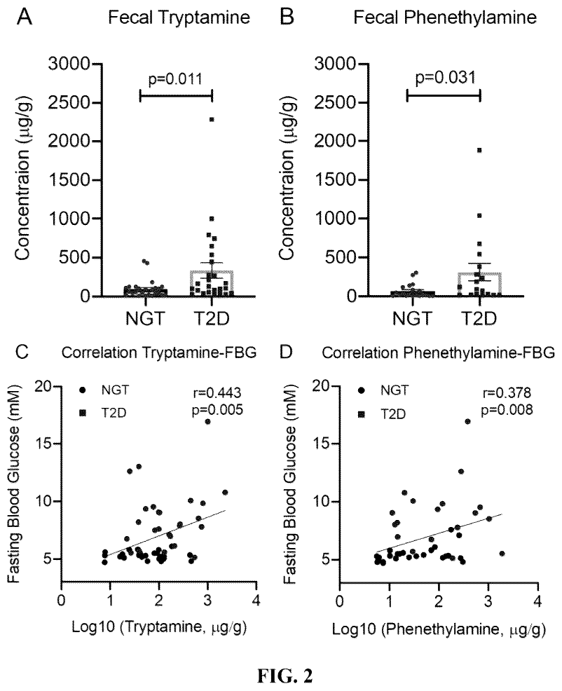

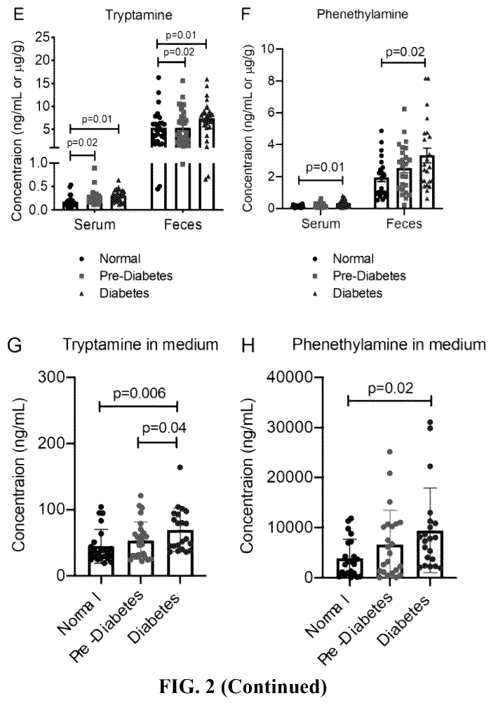

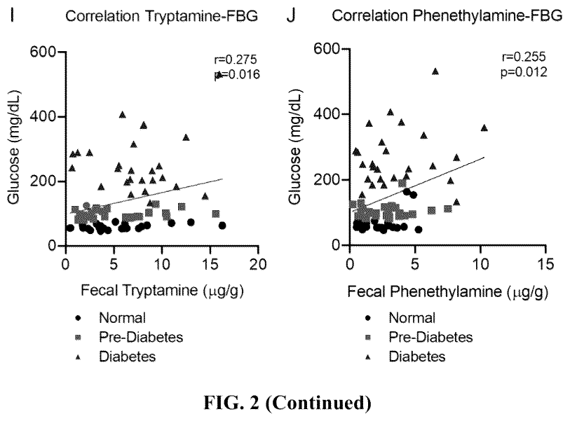

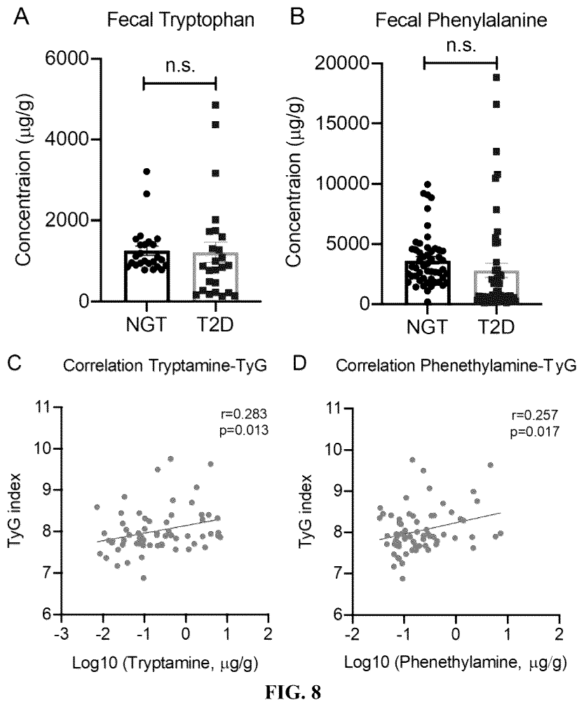

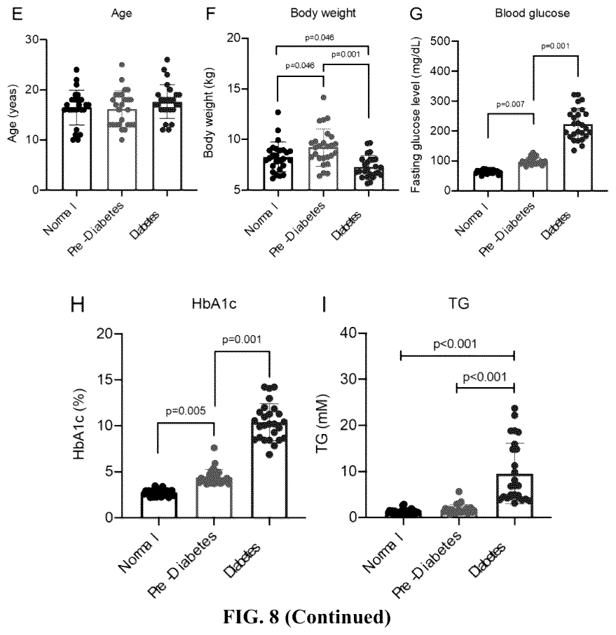

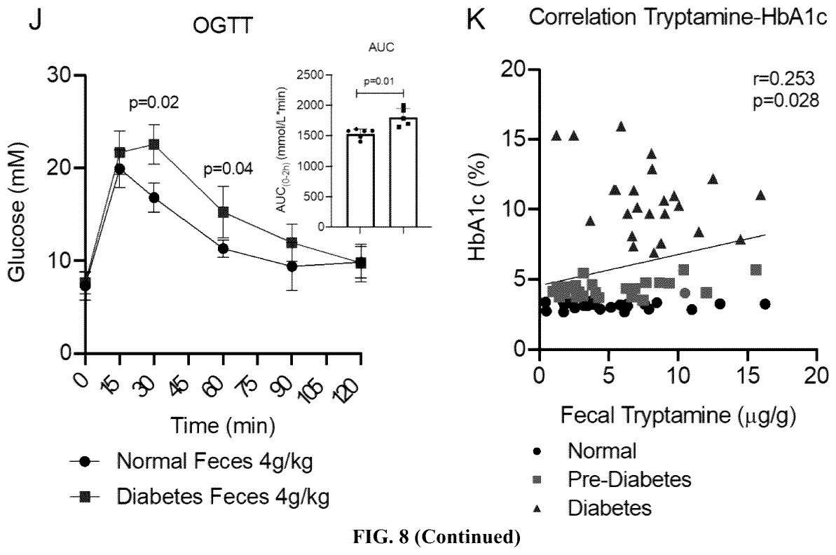

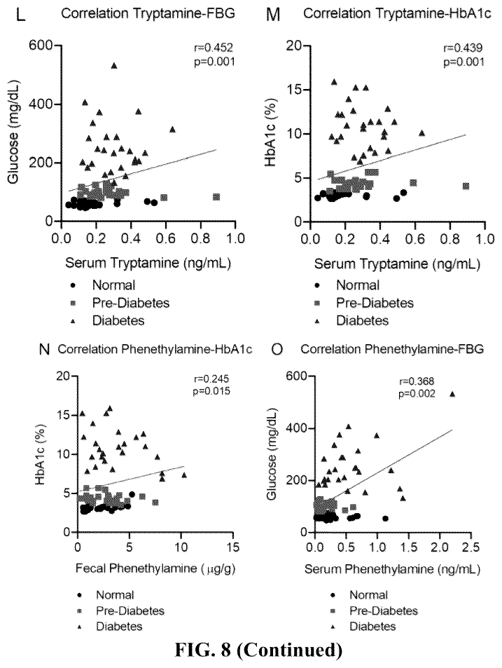

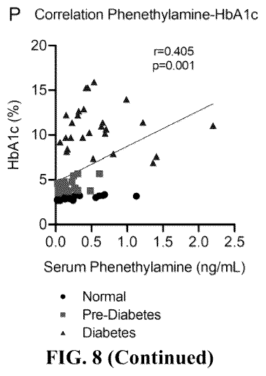

To investigate the clinical relevance of their findings obtained from animal studies, the fecal levels of tryptamine, phenethylamine and their precursors (tryptophan and phenylalanine) in healthy controls and T2D subjects were determined. It was found that tryptamine and phenethylamine were significantly higher in fecal samples from T2D subjects (p=0.011 and 0.031, FIG. 2A-B). In contrast, the levels of tryptophan and phenylalanine were not altered in fecal samples of T2D subjects (FIG. 8A-B). Correlation analyses revealed fecal tryptamine and phenethylamine are positively correlated with fasting blood glucose (FBG) levels (r=0.443 and 0.378, p=0.005 and 0.008, FIG. 2C-D) in T2D patients, revealing tryptamine and phenethylamine exhibit a positive association with glucose intolerance. The inventors then showed fecal tryptamine and phenethylamine levels were also positively correlated with TyG in healthy volunteers (r=0.283 and 0.257, p=0.013 and 0.017, FIG. 8C-D), suggesting that tryptamine and phenethylamine are potential indicators of metabolic syndrome.

Host variables, such as age, anti-diabetic medications, and dietary patterns, may confound gut microbiota studies of human diseases. To reduce the influence of these variables, the fecal tryptamine and phenethylamine levels in monkeys (Macaca fasicularis) with spontaneous metabolic syndrome, a pre-clinical primate model for metabolic diseases was determined. Based on FBG and HbA1c levels, age-matched and treatment-naive monkeys were assigned to normal group, pre-diabetes group or diabetes group (FIG. 8E-I). The inventors showed that mice fed with fecal suspensions from diabetic monkeys exhibited higher glucose levels than mice with fecal suspensions from normal monkeys (p<0.05 in all cases, FIG. 8J), suggesting a causal role of gut microbiota and their metabolic products in the development of glucose intolerance in primates.

The inventors then quantified tryptamine, phenethylamine and their precursors (tryptophan and phenylalanine) in the sera and feces of the monkeys. Consistently, tryptamine and phenethylamine, but not tryptophan and phenylalanine, were significantly increased in sera and feces of diabetic monkeys (p<0.05 in all cases, FIG. 2E-F). The inventors also found a higher concentration of tryptamine and phenethylamine in the culture medium of gut bacteria from fecal samples of pre-diabetic and diabetic monkeys under anaerobic conditions (p=0.006 and 0.02, FIG. 2G-H), confirming that diabetes-associated microbiota has a higher catalytic ability to transform tryptophan and phenylalanine into tryptamine and phenethylamine in monkeys. Correlation analyses revealed that fecal/serum levels of tryptamine and phenethylamine were all positively correlated with FBG and HbA1c in monkeys (r=0.275 and 0.255, p<0.02 in all cases, FIG. 2I-J and FIG. 8K-P).

### Tryptamine and Phenethylamine Impair Insulin Sensitivity

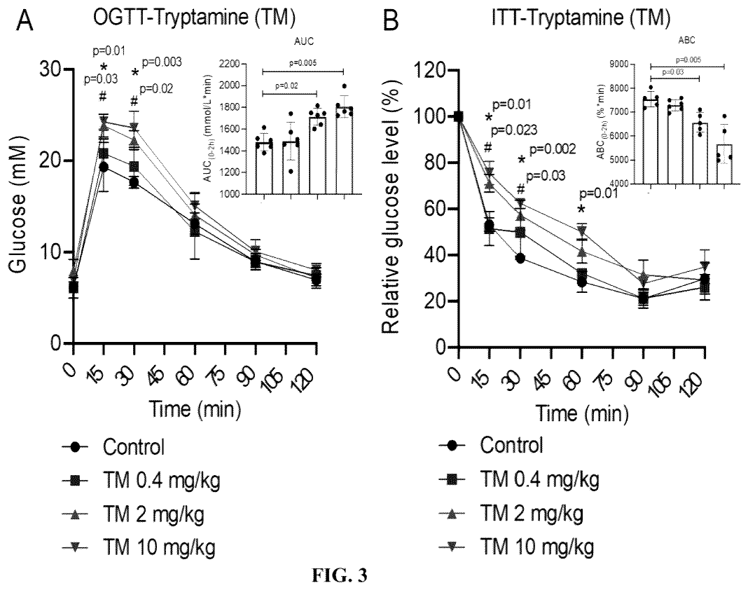

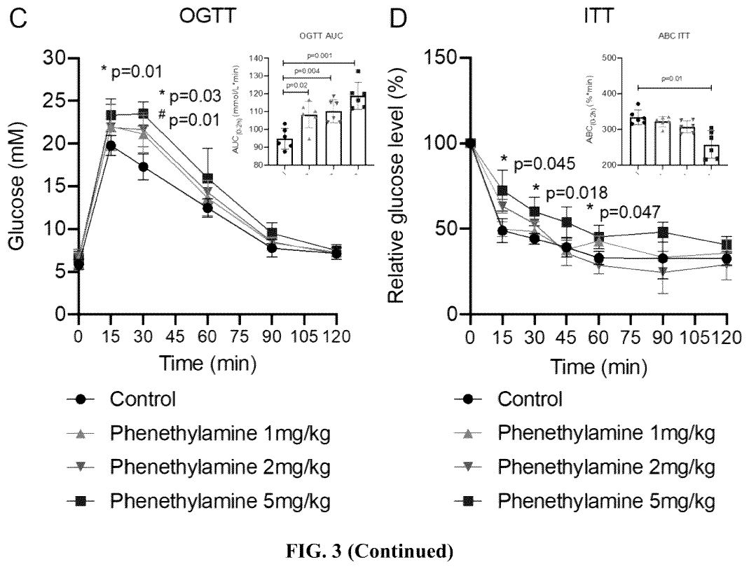

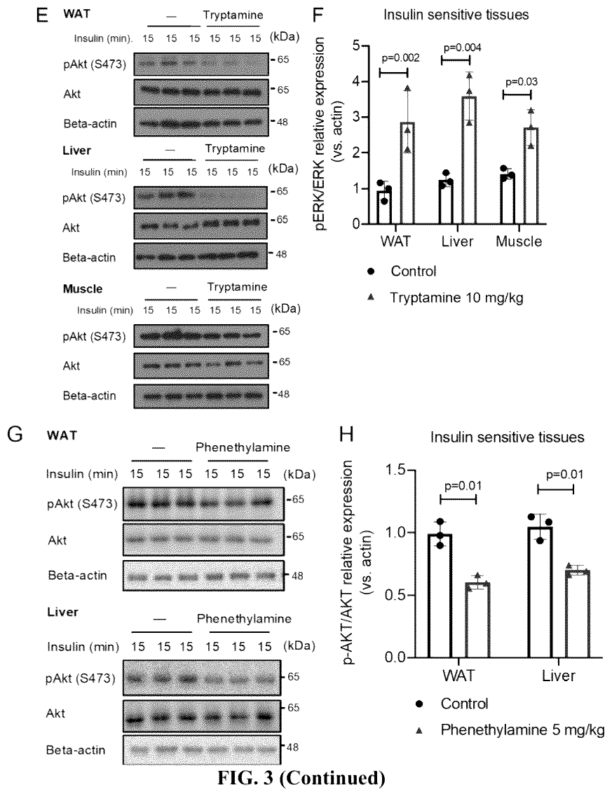

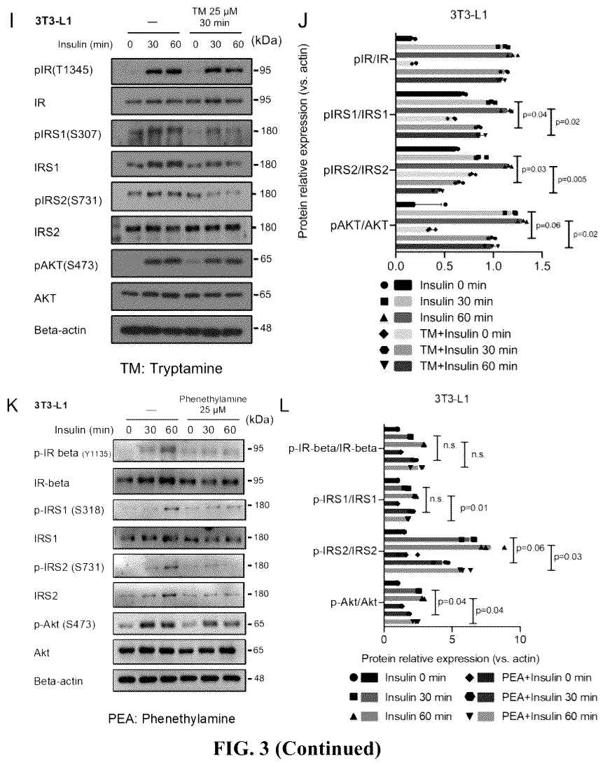

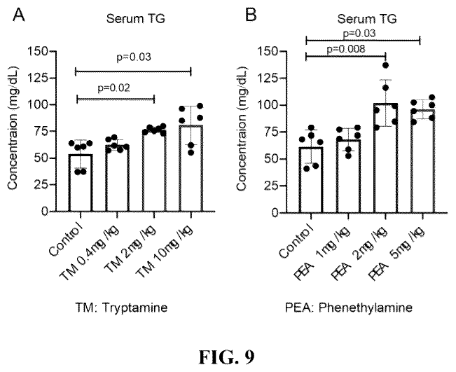

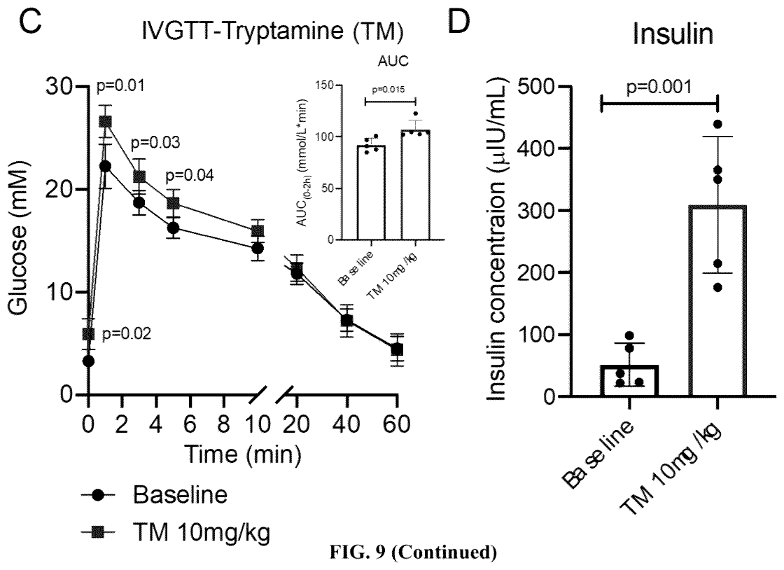

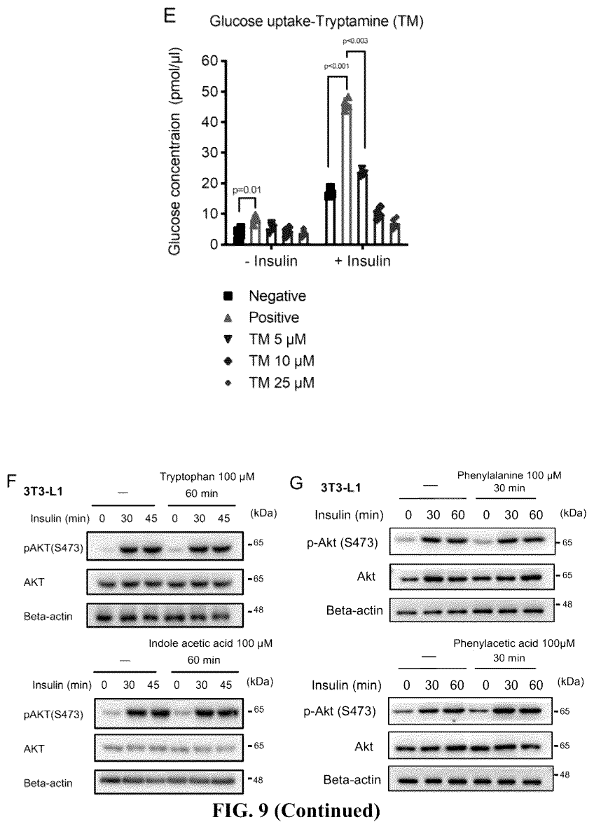

To understand the role of tryptamine and phenethylamine in the pathophysiology of insulin resistance, tryptamine and phenethylamine were administered to normal mice within the pathophysiological range detected in the fecal samples of T2D subjects. In normal mice intraperitoneally injected with tryptamine or phenethylamine it was found remarkably inhibitory effects of tryptamine and phenethylamine on glucose tolerance and insulin sensitivity observed by OGTT and insulin tolerance test (ITT) (p<0.05 in all cases, FIG. 3A-D). Moreover, tryptamine and phenethylamine significantly suppressed the insulin-induced Akt phosphorylation in major metabolic tissues, including white adipose tissue (WAT), liver and skeletal muscle (p<0.05 in all cases, FIG. 3E-H). It was also shown that tryptamine and phenethylamine induced a significant elevation of serum TG levels in normal mice (p<0.05 for both, FIG. 9A-B). To validate these findings in mice, an intravenous glucose tolerance test (IVGTT) was used to determine whether glucose tolerance was impaired by tryptamine in normal monkeys at a single dose of 10 mg/kg via oral gavage. Consistently, they showed tryptamine-treated monkeys exhibited higher blood glucose levels in the glucose challenge (p<0.05, FIG. 9C) and a significant elevation of serum insulin levels (p=0.001, FIG. 9D).

Following in vivo studies, the impact of tryptamine and phenethylamine on insulin signaling was also assessed in vitro. Treatment of tryptamine and phenethylamine inhibited insulin signaling in a time-dependent manner in 3T3-L1 adipocytes, a valid cell line for the study of insulin signaling (p<0.05 in all cases, FIG. 3I-L). The inventors also showed tryptamine inhibited basal glucose uptake in a dose-dependent manner in 3T3-L1 adipocytes (p<0.05 in all cases, FIG. 9E). In contrast, treatment with precursors and metabolites of tryptamine and phenethylamine including tryptophan, phenylalanine, indole-3-acetic acid and phenylacetic acid in similar doses did not alter the insulin-induced AKT phosphorylation in 3T3-L1 cells (FIG. 9F-G), suggesting that tryptamine and phenethylamine, but not their precursors or metabolites, impaired glucose tolerance and insulin sensitivity.

### Tryptamine and Phenethylamine Weaken Insulin Signaling Via TAAR1-ERK Activation

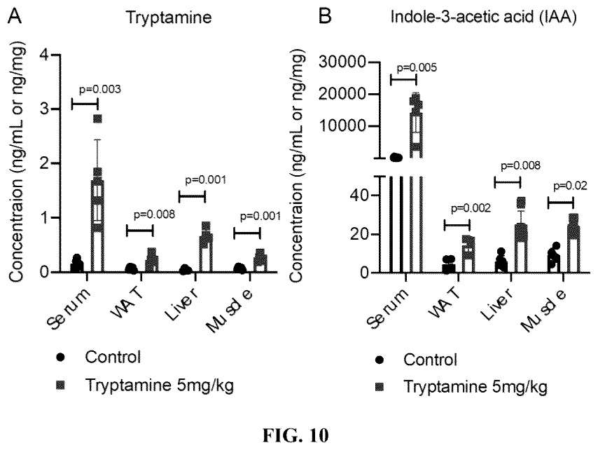

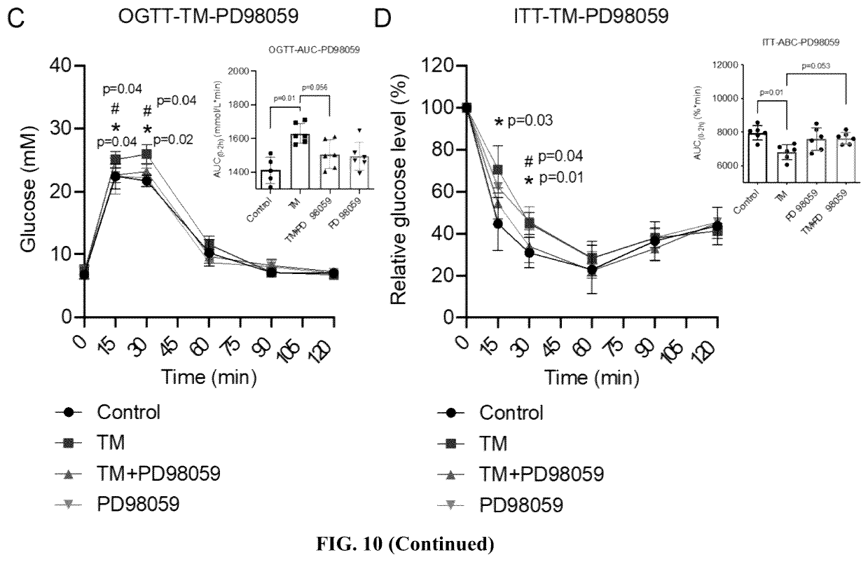

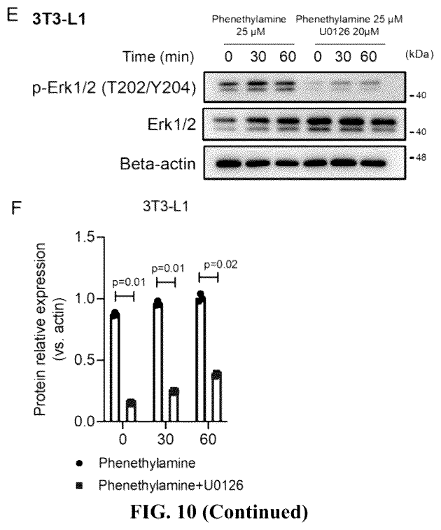

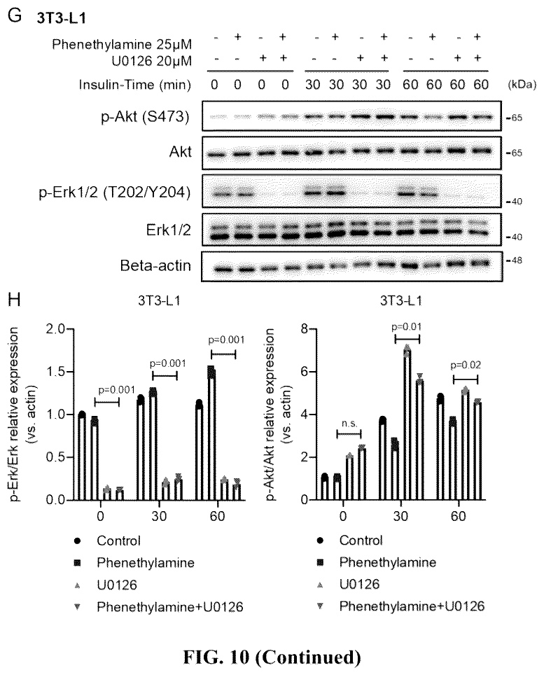

Tryptamine and phenethylamine can bind to and activate GPCR receptor TAAR1, which may exert inhibitory effects on the downstream insulin signaling pathway. After oral gavage of tryptamine, we observed that tryptamine and its metabolite indole-3-acetic acid (IAA) levels were significantly increased in serum and insulin-sensitive tissues within 15 min (p<0.05 in all cases, FIG. 10A-B), suggesting that tryptamine enters insulin-sensitive tissues and is simultaneously metabolized by the host after being produced by gut microbiota.

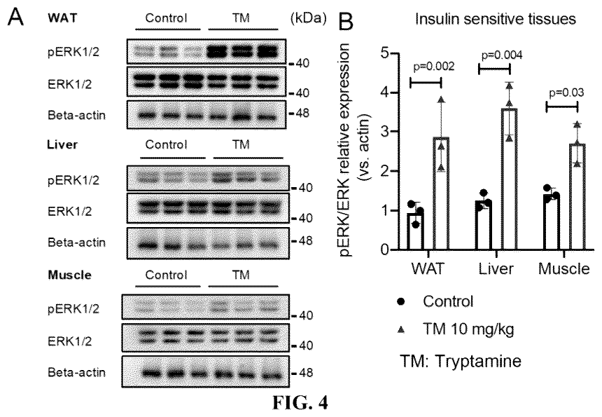

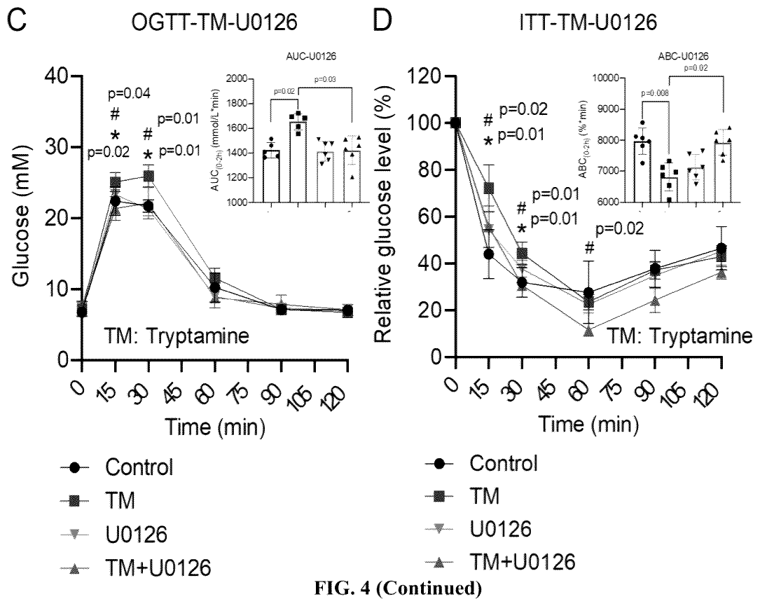

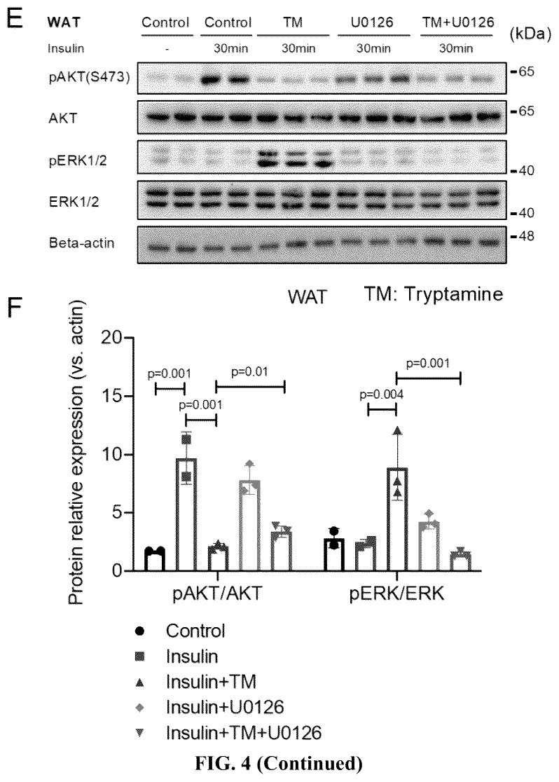

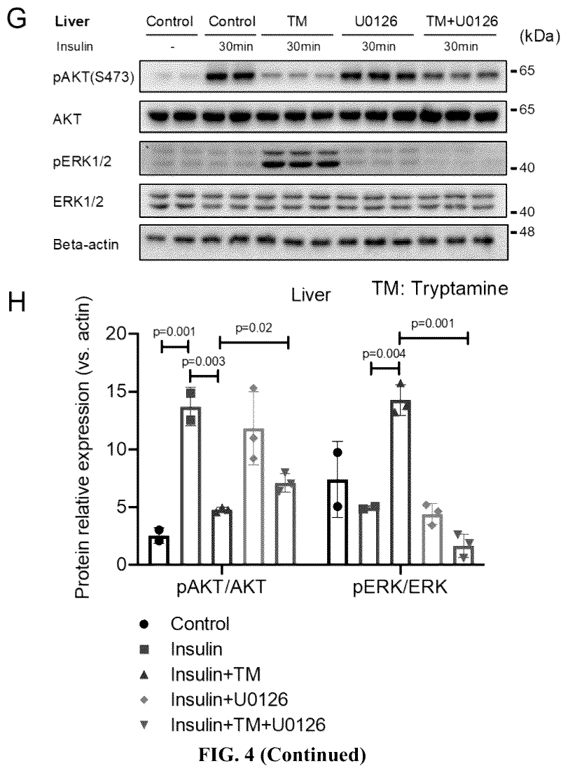

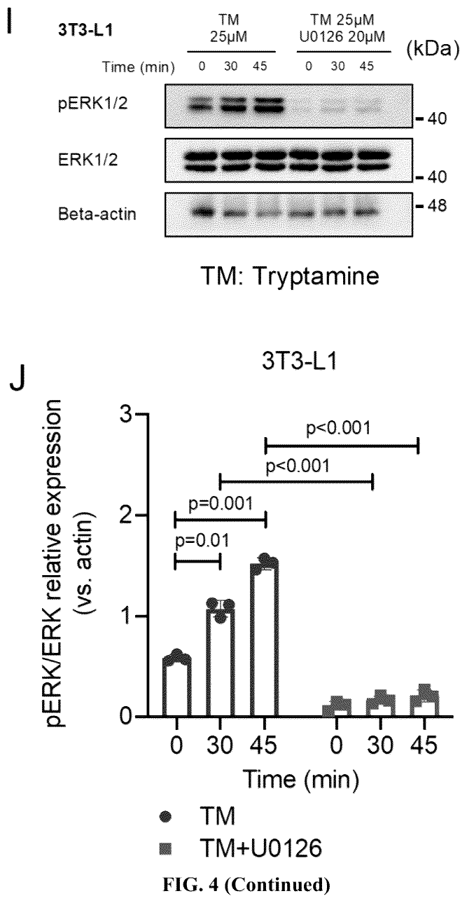

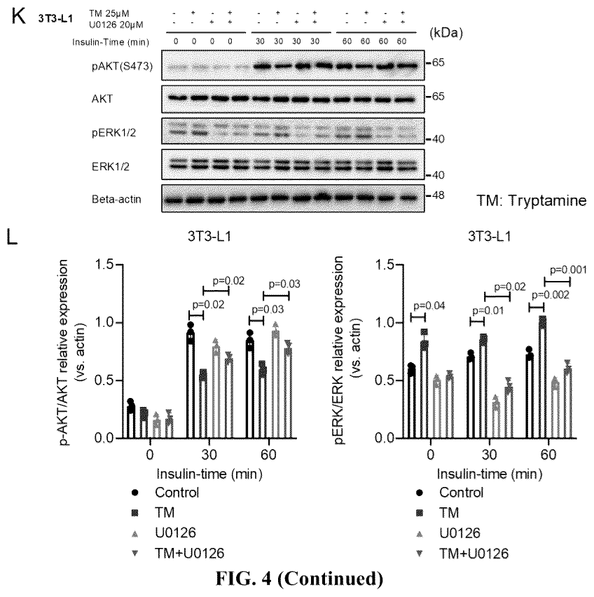

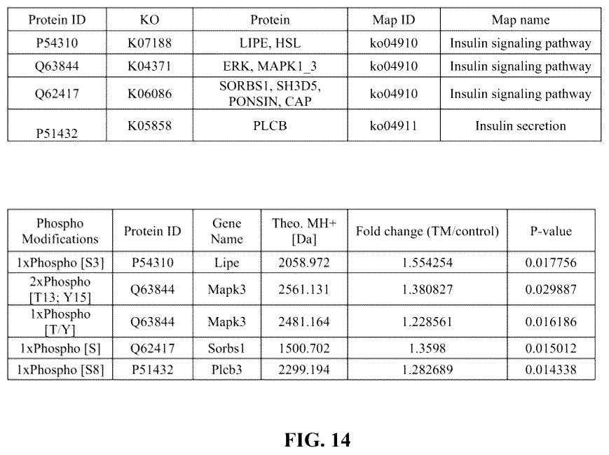

To investigate the mechanism(s) underlying tryptamine and phenethylamine inhibition on insulin signaling, a phospho-proteomics approach to capture the molecular components that are significantly altered by tryptamine was employed. Four insulin signaling-related proteins were found, including hormone-sensitive lipase (HSL), mitogen-activated protein kinase MAPK 1/3 (ERK), sorbin and SH3 domain containing 1 (SH3D5), were upregulated in insulin-sensitive tissues in response to tryptamine treatment (FIG. 14). Among these proteins, ERK has previously been implicated in the pathogenesis of IR in T2D. To test whether tryptamine and phenethylamine activate the MAPK/ERK pathway to suppress insulin sensitivity, ERK1/2 phosphorylation in tryptamine-treated mice (i.p.) was examined and it was found that ERK1/2 phosphorylation is upregulated in insulin-sensitive tissues by tryptamine (p<0.05 in all cases, FIG. 4A-B). Next, ERK inhibitors (U0126 and PD98059) were used to determine whether the suppressive effect of tryptamine on glucose tolerance and insulin sensitivity is dependent on the MAPK/ERK pathway. In OGTT and ITT studies, treatment with ERK inhibitors significantly improved glucose intolerance and insulin resistance in tryptamine-treated mice (p<0.05 in all cases, FIG. 4C-D and FIG. 10C-D). By contrast, ERK inhibitors exerted negligible effects on glucose tolerance or insulin sensitivity in control mice. In addition, ERK inhibitors abolished the inhibitory effects of tryptamine on the insulin-stimulated AKT phosphorylation and significantly downregulated tryptamine-induced ERK phosphorylation in insulin-sensitive tissues (p<0.05 in all cases, FIG. 4E-H). In line with these in vivo observations, tryptamine and phenethylamine also induced ERK1/2 phosphorylation in a time-dependent manner in 3T3-L1 cells, which was blocked by pre-treatment with ERK inhibitors (p<0.05 in all cases, FIG. 4I-J and FIG. 10E-F). ERK inhibitors also significantly suppressed the inhibitory effects of tryptamine and phenethylamine on AKT phosphorylation in insulin-stimulated 3T3-L1 cells (p<0.05 in all cases, FIG. 4K-L and FIG. 10G-H). These results suggest that tryptamine and phenethylamine impair insulin sensitivity through activating the MAPK/ERK pathway.

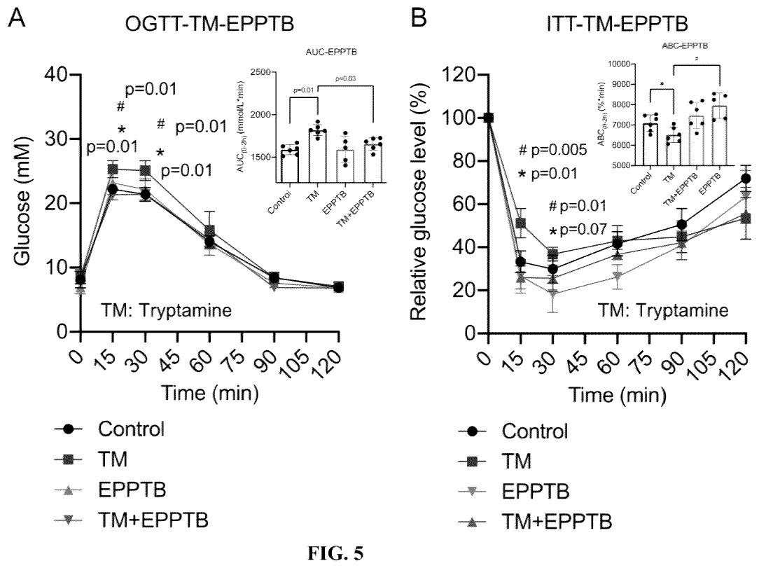

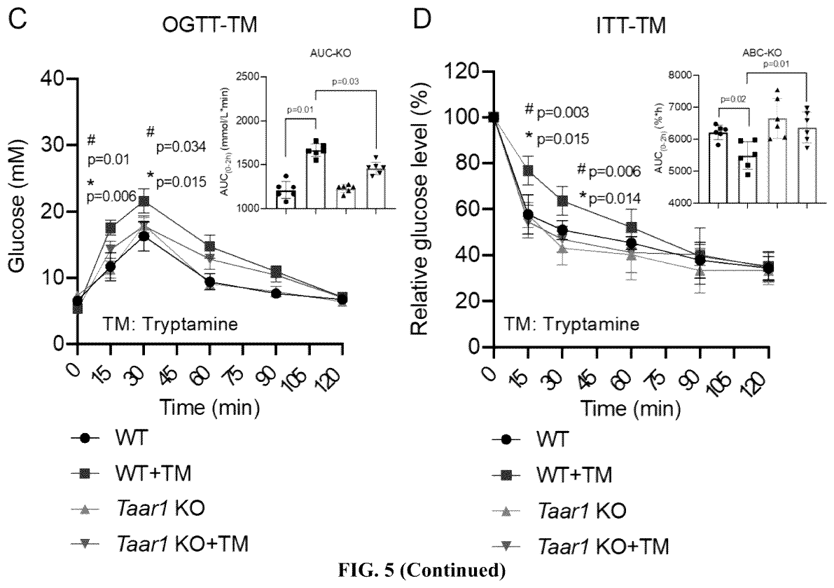

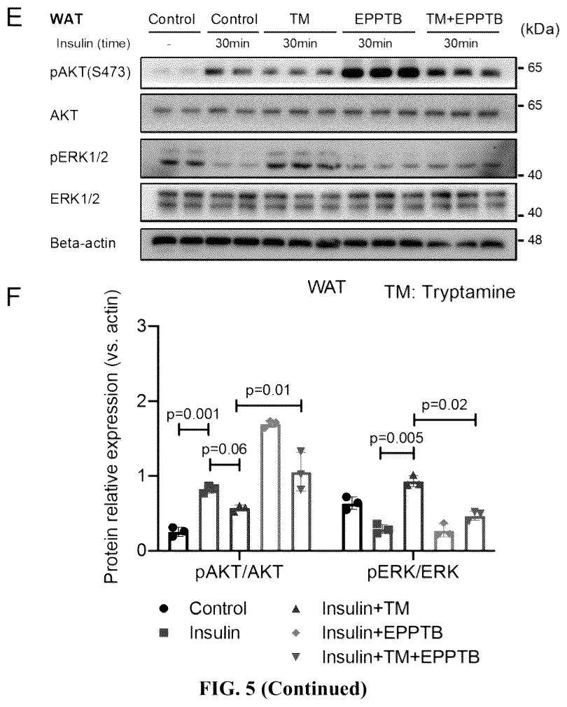

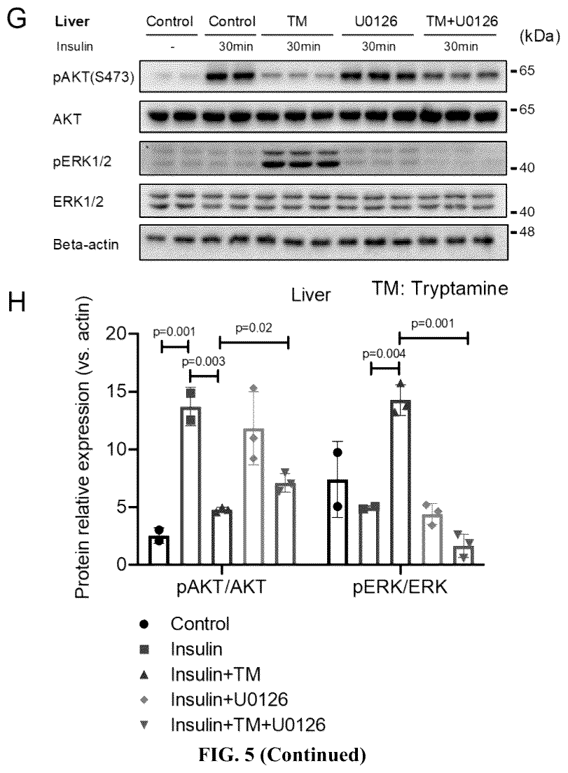

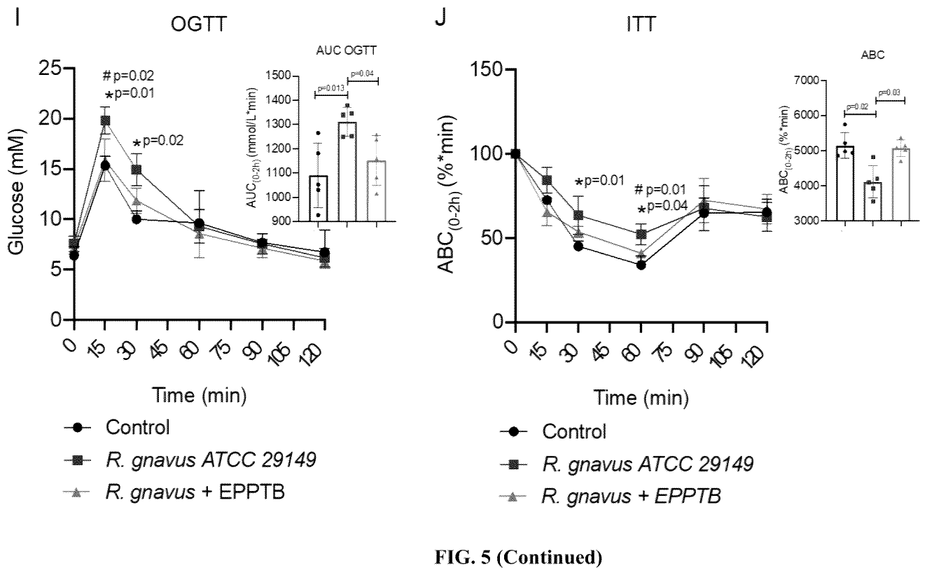

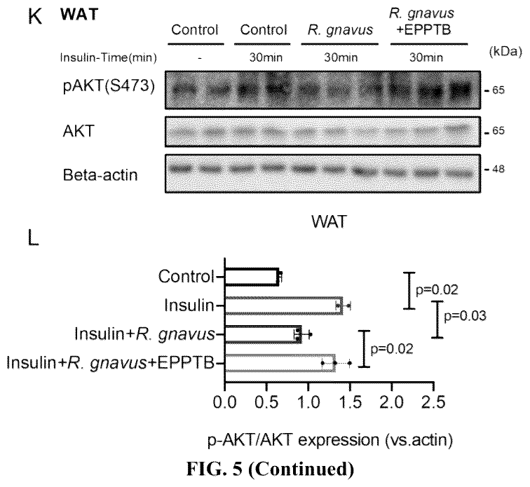

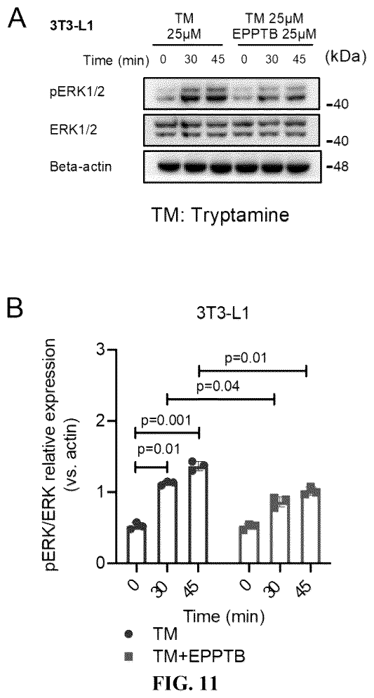

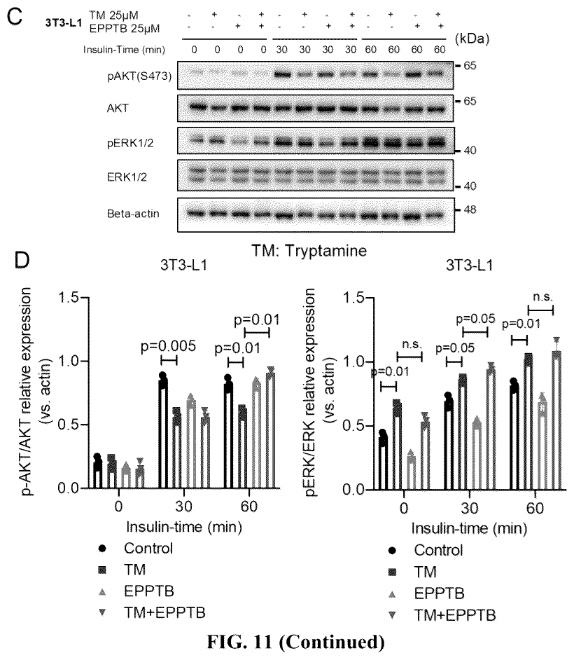

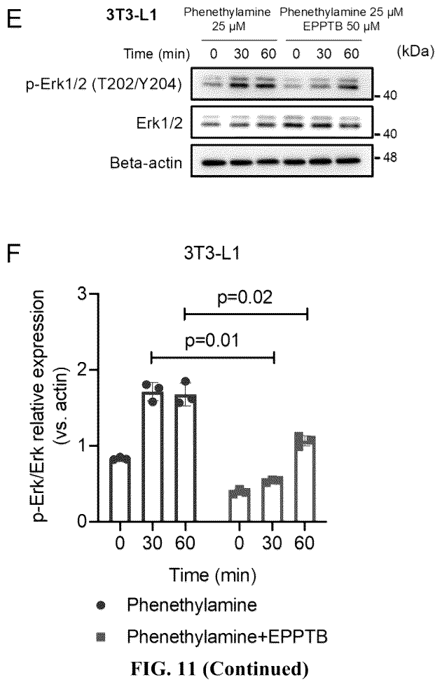

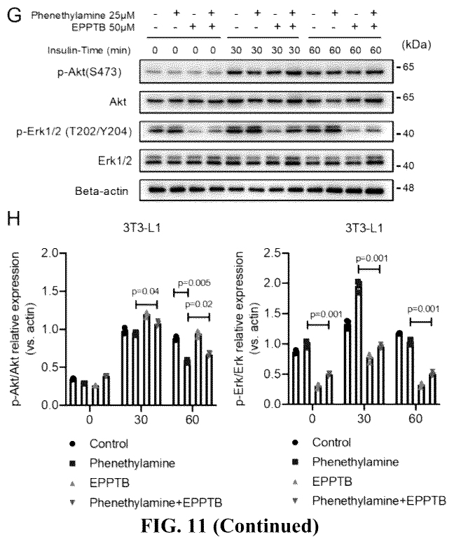

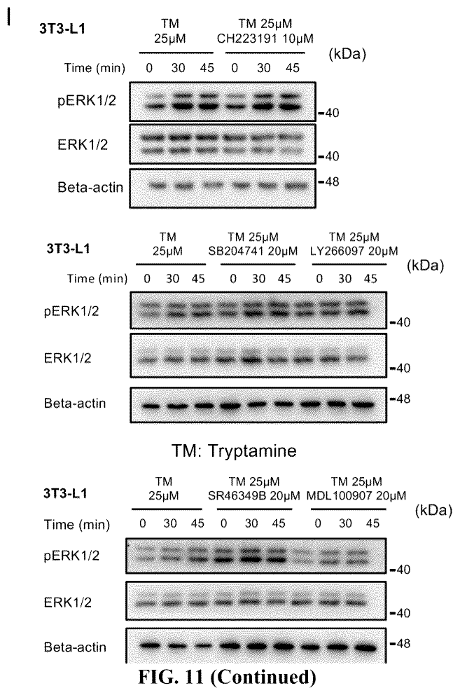

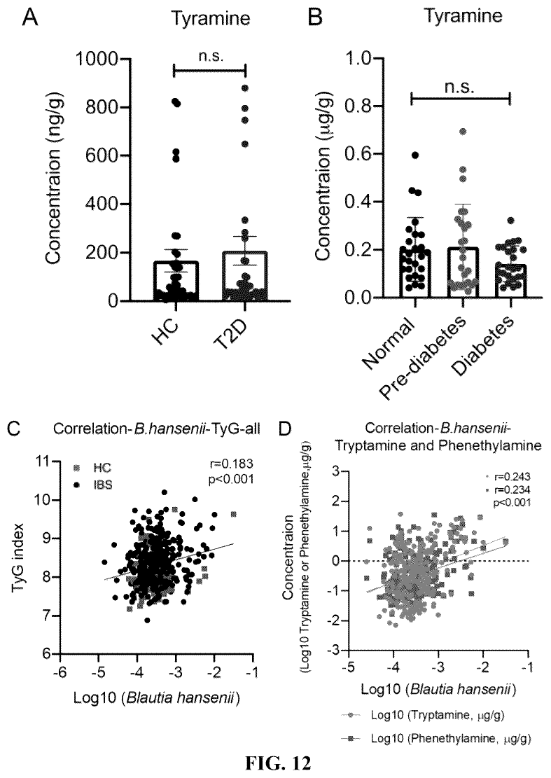

Furthermore, it was examined whether tryptamine and phenethylamine act through the TAAR1-MAPK/ERK signaling axis to inhibit insulin signaling. In the OGTT and ITT studies, treatment with EPPTB (a specific TAAR1 antagonist) or genetic ablation of Taar1 significantly reduced tryptamine-induced glucose intolerance and insulin resistance in mice (p<0.05 in all cases, FIG. 5A-D). TAAR1 antagonism by EPPTB also downregulated tryptamine-induced ERK phosphorylation and abolished the inhibitory effects of tryptamine on insulin-stimulated AKT phosphorylation (p<0.05 in all cases, FIG. 5E-G). In contrast, EPPTB did not affect either glucose tolerance nor insulin sensitivity in control mice. In line with these in vivo results, EPPTB treatment also significantly downregulated the increased ERK phosphorylation induced by tryptamine and phenethylamine and reversed the inhibitory effects of tryptamine and phenethylamine on insulin-stimulated AKT phosphorylation in 3T3-L1 cells (p<0.05 in all cases, FIG. 11A-H).

It was then investigated whether TAAR1 inhibition blocks R. gnavus action on glucose tolerance and insulin signaling in antibiotics-treated mice. Antibiotics-treated mice colonized with R. gnavus exhibited lower insulin sensitivity and impaired glucose tolerance. In contrast, these insulin-desensitizing effects of R. gnavus were partially abrogated by EPPTB treatment (p<0.05 in all cases, FIG. 5I-L), indicating inhibition of TAAR1 alleviated R. gnavus-induced insulin resistance.

R. gnavus, Tryptamine and Phenethylamine are Negatively Correlated with the Improvement of Insulin Sensitivity in a Dietary Fiber Intervention Study

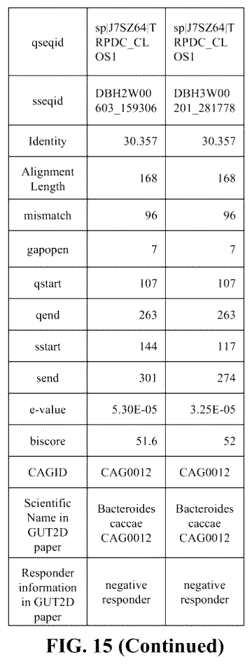

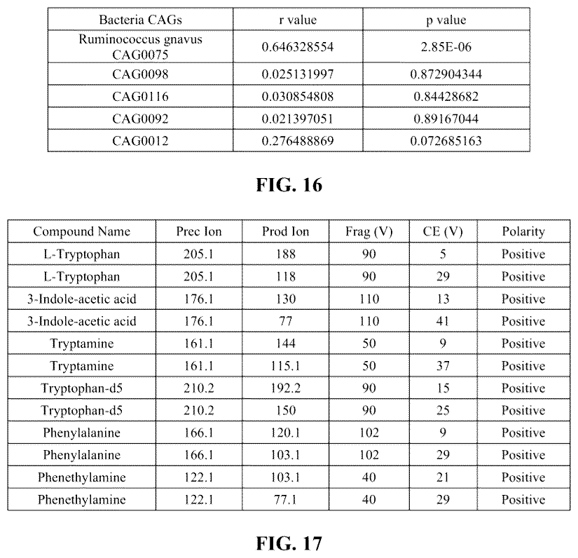

Previous studies have reported that dietary fiber intervention improved glucose homeostasis in T2D subjects by promoting the growth of short-chain fatty acids-producing bacteria. Interestingly, R. gnavus CAG0075 significantly suppressed dietary fiber intervention in a clinical study (FIG. 16). The TDC sequence of R. gnavus CAG0075 had 100% identity compared with the reference TDC sequence from R. gnavus (ATCC 29149) and the average nucleotide identity between R. gnavus CAG0075 and R. gnavus (ATCC 29149) was 99.1%, highlighting the R. gnavus TDC is negatively correlated with the improvement of insulin sensitivity in T2D subjects.

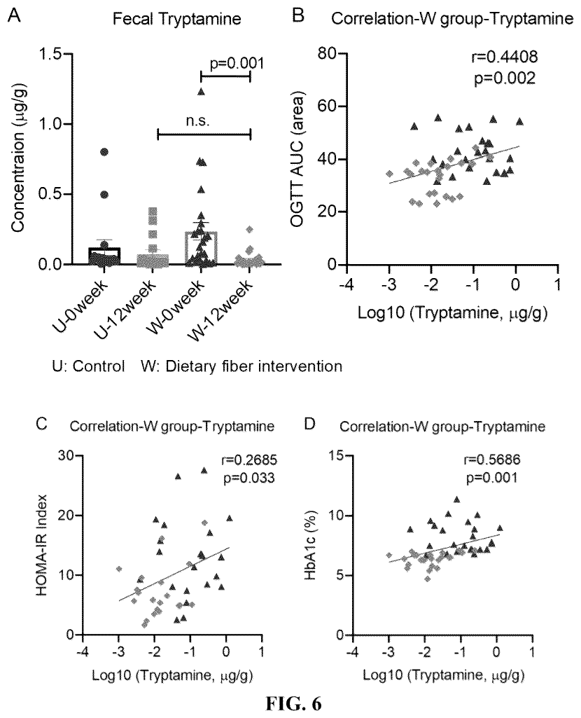

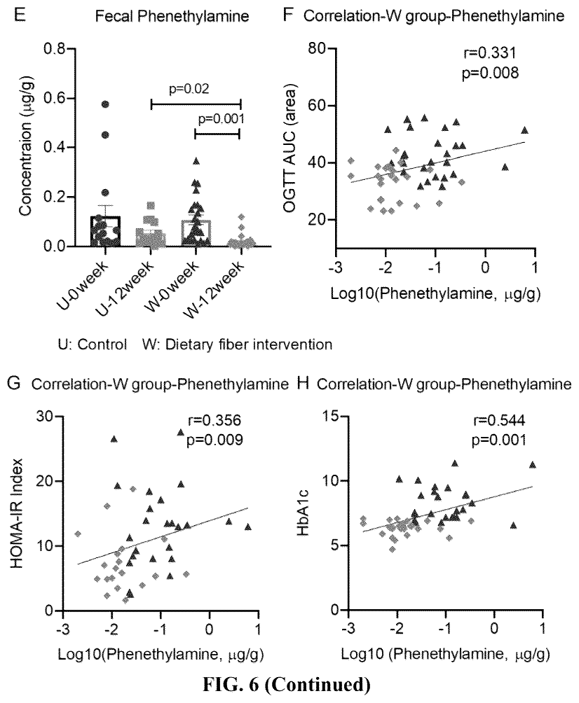

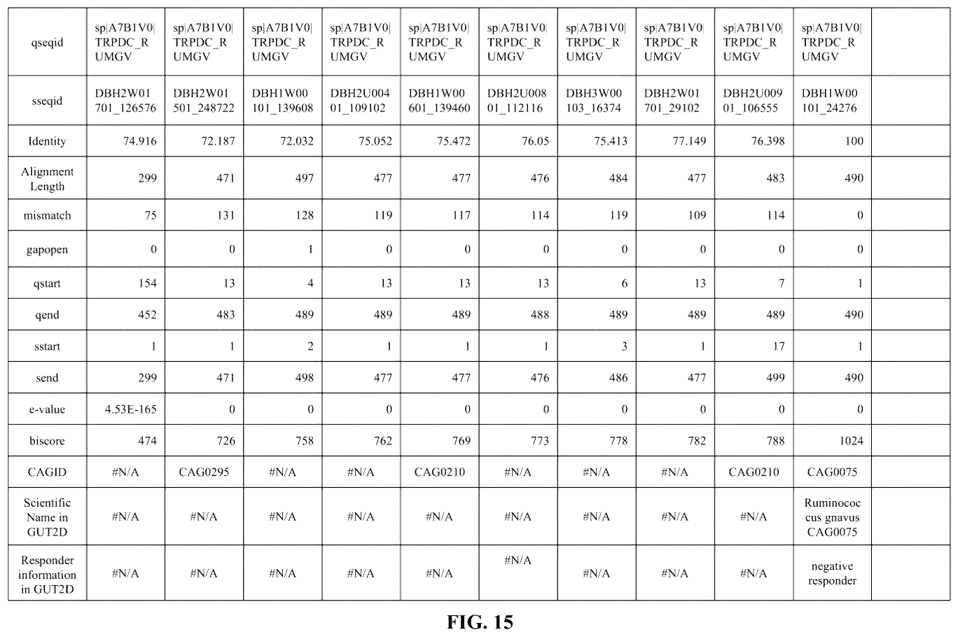

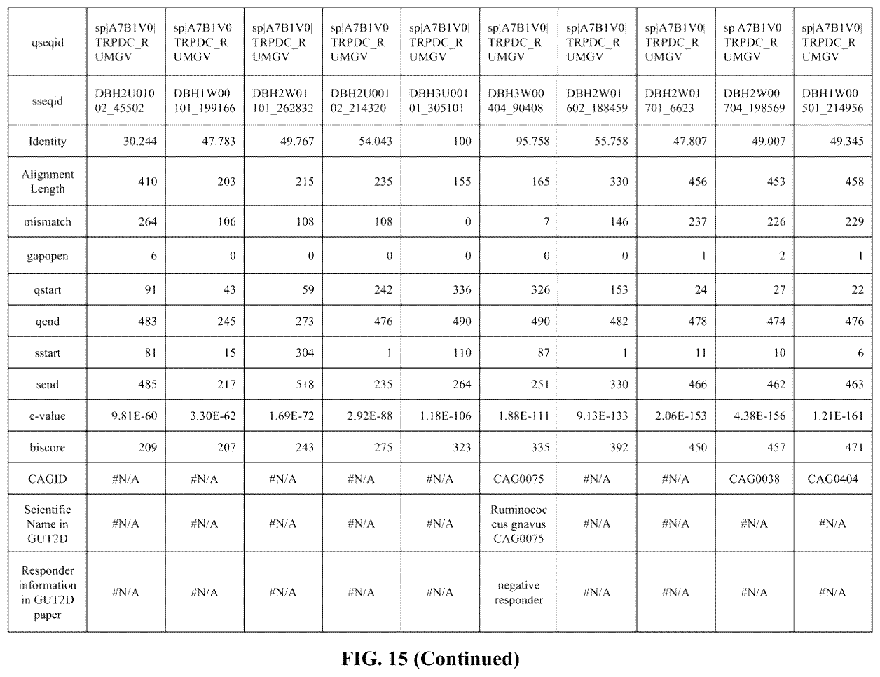

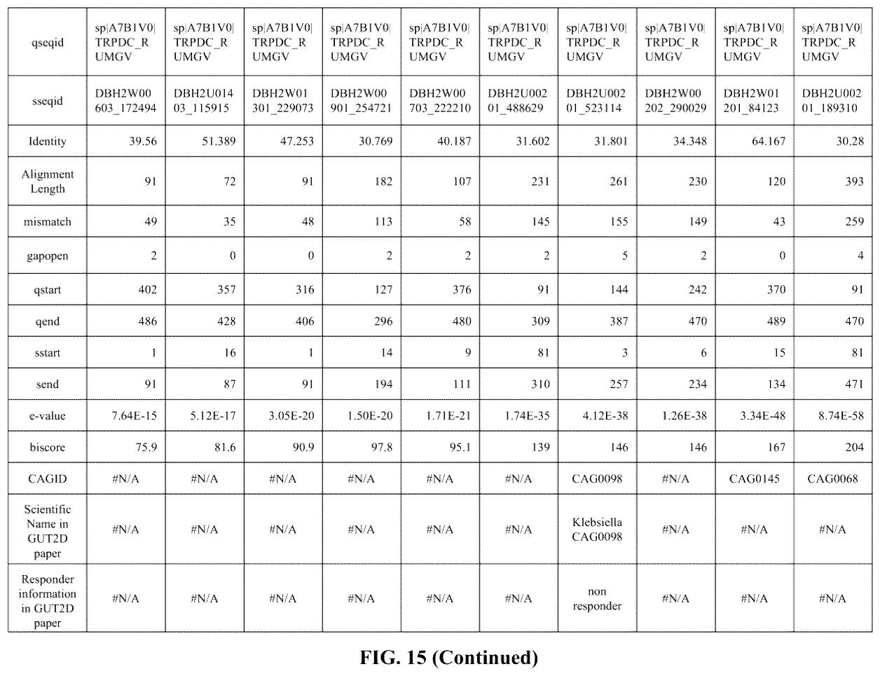

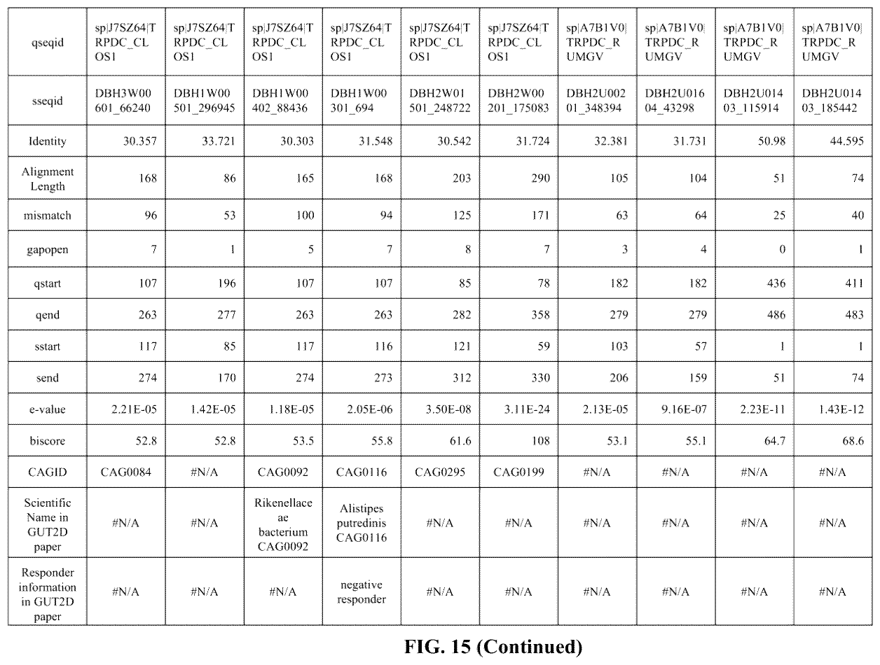

Tryptamine and phenethylamine levels in fecal samples of T2D subjects who consumed a high-fiber diet in this study were then determined. Consistent with the reduction of R. gnavus, fecal tryptamine and phenethylamine levels were significantly suppressed by the dietary fiber intervention (as shown in W group) (p=0.001, FIG. 6A and E). In the high dietary fiber-treated T2D subjects (W group), downregulation of fecal tryptamine and phenethylamine levels were found positively correlated with improvements seen in OGTT, HbA1c and Homeostatic model assessment for insulin resistance (HOMA-IR) indexes (r=0.441, 0.569 and 0.269, and r=0.325, 0.487 and 0.286 after adjustment to BMI, p<0.05 in all cases, FIG. 6B-D and FIG. 6F-H). In addition, correlation analyses between tryptamine/phenethylamine and bacterial genomes with the TDC sequence showed that only R. gnavus CAG0075 is positively correlated with tryptamine among the 5 genomes with the TDC genes (FIG. 15). These results suggest that the R. gnavus accompanied with tryptamine and phenethylamine are negatively correlated with improvement of insulin sensitivity in T2D subjects.

Growing evidence suggests a causal link between the gut microbiome and human metabolic health. A recent cross-sectional study (n=5215 in total) showed R. gnavus has the strongest association with features of metabolic syndrome among 50 identified prevalent gut microbes in species-level. However, the causal effects of R. gnavus on metabolic syndrome and its pathogenetic mechanisms have not been explored yet. The present disclosure demonstrates tryptamine/phenethylamine-mediated TAAR1 signaling pathway as the key molecular axis underlying R. gnavus-induced insulin resistance is ofutmost importance, paving the way for the development of better treatment for metabolic syndrome induced by gut dysbiosis. Colonization of R. gnavus impaired insulin sensitivity along with significantly increased levels of tryptamine and phenethylamine, suggesting a potential pathogenic role of R. gnavus-derived tryptamine and phenethylamine in the development of insulin resistance. Furthermore, not only human subjects but also a pre-clinical monkey model of metabolic syndrome were involved to address the correlation between tryptamine/phenethylamine and insulin resistance for several reasons. First, unlike diet and chemical-induced or genetically induced animal models of T2D, the monkeys spontaneously develop metabolic syndrome that is characterized by hyperglycemia, hyperlipidemia and insulin resistance. Second, the studies involving experimental monkeys are not influenced by confounding variables that can affect the gut microbiome in human studies, such as age, anti-diabetic medications and dietary patterns. Gut microbiome composition may vary considerably across geographic locations, races and ethnicities, while gut-microbial metabolites profiles are highly conserved, suggesting the use of the combined application of gut microbes and gut-microbial metabolites in the prognosis and diagnosis of human diseases.

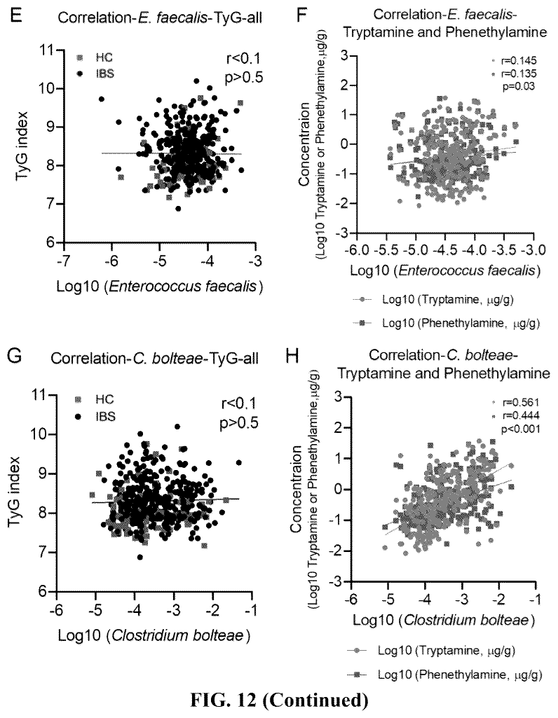

From a mechanism of action standpoint, it was revealed tryptamine and phenethylamine derived from the catabolism of R. gnavus on dietary amino acids impaired insulin sensitivity via activation of TAAR1-MAPK/ERK signaling pathway axis, thereby contributing to insulin resistance in metabolic syndrome and T2D. TAAR1 is an amine-activated G protein-coupled receptor that is activated by gut microbes-derived aromatic trace amines including tryptamine, phenethylamine and tyramine in gut. In contrast, it was shown only tryptamine and phenethylamine but not tyramine was altered in diabetic monkeys and T2D patients (FIG. 12 A-B). A TAAR1 exogenous activator has recently been proposed as a potential target for improving glycemic control and TAAR1/Gas-mediated signaling pathways promote insulin secretion in beta-cells. However, the present disclosure shows long-term exposure to endogenous TAAR1 activators tryptamine and phenethylamine impair insulin sensitivity, thereafter the pharmacological use of TAAR1 modulator on glucose control should be carefully considered in terms of dose and route of administration. MAPK/ERK signaling pathway is involved in the development of insulin resistance associated with metabolic syndrome and T2D. ERK activity is elevated in WAT of humans and rodents in diabetic conditions and activation of the ERK signaling pathway can significantly reduce the expression of key mediators of insulin signaling. In addition, inhibition of the ERK pathway using specific chemical inhibitors is effective in alleviating insulin resistance in db db and High fat diet (HFD)-fed mice. It was demonstrated tryptamine and phenethylamine actions on ERK activation and inhibition of insulin signaling pathway can be abolished by TAAR1 antagonist and genetic ablation of Taar1, representing targeting TAAR1-MAPK/ERK signaling axis as a potential therapeutic strategy to combat R. gnavus-induced insulin resistance.

Besides TAAR1, several receptors of tryptamine including aryl hydrocarbon receptor (AhR) and 5-HTR₄ have also been identified. However, tryptamine-mediated activation of ERK was not suppressed by antagonists to either AhR or 5-HTR₄ in 3T3-L1 cells, suggesting TAAR1 plays a predominant role (FIG. 11I). Although tryptamine and its metabolite indole-3-acetic acid (IAA) are agonists of AhR signaling, the beneficial effects of AhR activation on the control of IR are likely predominantly mediated by IAA as the IAA concentration is about 40-100-fold to that of tryptamine in serum (FIG. 10A). Importantly, the findings revealed that IAA treatment did not impair glucose tolerance nor insulin sensitivity in normal mice (FIG. 9F), suggesting that the metabolic dysfunctions induced by alterations in the composition of the gut microbiome are primarily mediated by the tryptamine/TAAR1 signaling axis but not the IAA/AhR signaling axis in the context of T2D. This suggests that tryptamine and phenethylamine as TAAR1 ligands are the culprits and one possible way to design a therapeutic approach is to facilitate the reduction of R. gnavus or on top of blocking the tryptamine and phenethylamine production by inhibiting the bacterial TDC.

Besides the mechanistic insights into the contribution of R. gnavus-derived tryptamine and phenethylamine to the development of insulin resistance, a variety of bacteria species including Blautia hansenii, Enterocloster (Clostridium) boltae and Enterococcus faecalis and have also been shown to produce tryptamine and phenethylamine by recent studies. Among these tryptamine and phenethylamine-producing bacteria species, R. gnavus has the highest catalytic ability to transform aromatic amino acids into aromatic trace amines (tryptamine, tyramine and phenethylamine) compared with other bacteria species. To further uncover the role of tryptamine and phenethylamine-producing gut bacteria that is involved in the development of insulin resistance, the inventors also investigated the associations between these bacteria, tryptamine/phenethylamine and indicators of insulin resistance in their participants (FIG. 12C-H), providing supporting evidence for the increased abundances of tryptamine and phenethylamine producers in metabolic syndrome. The implications of these bacterial strains in metabolic syndrome will be studied further. Given bacterial TDC exists among many bacterial strains, the therapeutic approach to regulate tryptamine and phenethylamine levels either by reducing the abundance of tryptamine and phenethylamine producers such as R. gnavus or inhibiting tryptophan decarboxylase or blockade of tryptamine/TAAR1 signaling might be feasible.

Interestingly, the present disclosure shows dietary fiber intervention significantly suppressed tryptamine and phenethylamine levels and abundances of R. gnavus in T2D subjects, revealing manipulation of gut microbiota-derived tryptamine and phenethylamine by dietary changes or prebiotics is a new direction for managing metabolic syndrome. It was shown that R. gnavus reduction accompanied by downregulation of tryptamine and phenethylamine are positively correlated with the improvement of insulin resistance. Given the new insights on the interactions between foods, the gut microbiota and metabolic homeostasis, managing diet from a systemic perspective to reduce the risks of developing metabolic diseases may become an important therapeutic strategy. Further details about the mechanism are worthy to identify the proper management protocol from diet aspects to modulate the R. gnavus abundance and tryptamine and phenethylamine levels.

In summary, the data showed that R. gnavus and its derived tryptamine and phenethylamine are important factors in the pathogenesis of insulin resistance in metabolic syndrome and T2D.

What is claimed is:

1. A method of treating a metabolic disorder in a subject in need thereof, the method comprising: providing a fecal sample obtained from the subject; determining the amount of one or more markers selected from the group consisting of phenethylamine, tryptamine, and Ruminococcus gnavus in the fecal sample; determining based on the amount of the one or more markers in the fecal sample that the subject has the metabolic disorder; and administering a therapeutically effective amount of a trace amine-associated receptor 1 (TAAR1) inhibitor to the subject.

2. The method of claim 1, wherein the metabolic disorder is selected from the group consisting of insulin resistance, glucose intolerance, hyperglycemia, hyperlipidemia, pre-diabetes, type 2 diabetes, and combinations thereof.

3. The method of claim 1, wherein the metabolic disorder comprises insulin resistance.

4. The method of claim 1, wherein the TAAR1 inhibitor selectively binds TAAR1.

5. The method of claim 1, wherein the TAAR1 inhibitor is a small molecule.

6. The method of claim 1, wherein the TAAR1 inhibitor is a compound of Formula 1: 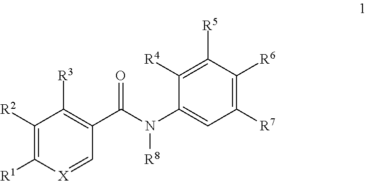
or a pharmaceutically acceptable salt thereof, wherein
n is 0, 1, 2 or 3;
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;
X is —C(R⁹)═ or —N═;
R⁹ is hydrogen, C₁-C₆ alkoxy, NO₂, or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

7. The method of claim 1, wherein the TAAR1 inhibitor is N-(3-ethoxy-phenyl)-4-pyrrolidin-1-yl-3-trifluoromethyl-benzamide.

8. The method of claim 1, wherein the subject is a human, a non-human primate, a rodent, a canine, a feline, a bovine, or an equine.

9. The method of claim 1, wherein the subject is a human.

10. The method of claim 1, wherein the metabolic disorder comprises insulin resistance and the one or more markers comprises Ruminococcus gnavus.

11. The method of claim 1, wherein the step of determining based on the amount of the one or more markers in the fecal sample that the subject has the metabolic disorder comprises comparing the amount of the one or more markers in the fecal sample with an average amount of the one or more markers in fecal samples obtained from healthy controls.

12. The method of claim 1, wherein the amount of the one or more markers in the fecal sample obtained from the subject is higher than the average amount of the one or more markers in fecal samples obtained from healthy controls.

13. A method of treating insulin resistance in a subject in need thereof, the method comprising: providing a fecal sample obtained from the subject; determining the amount of Ruminococcus gnavus in the fecal sample; determining based on the amount of Ruminococcus gnavus in the fecal sample that the subject has the metabolic disorder; and administering a therapeutically effective amount of a trace amine-associated receptor 1 (TAAR1) inhibitor to the subject.

14. The method of claim 13, wherein the TAAR1 inhibitor is a compound of Formula 1: 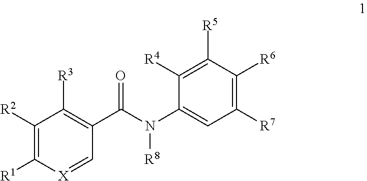
or a pharmaceutically acceptable salt thereof, wherein
n is 0, 1, 2 or 3;
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;
X is —C(R⁹)═ or —N═;
R⁹ is hydrogen, C₁-C₆ alkoxy, NO₂, or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

15. The method of claim 13, wherein the TAAR1 inhibitor is N-(3-ethoxy-phenyl)-4-pyrrolidin-1-yl-3-trifluoromethyl-benzamide.

16. A method of diagnosing a metabolic disorder in a subject, the method comprising providing a fecal sample obtained from the subject; determining the amount of one or more markers selected from the group consisting of Ruminococcus gnavus, phenethylamine, and tryptamine in the fecal sample; determining based on the amount of the one or more markers in the fecal sample if the subject has the metabolic disorder.

17. The method of claim 16, wherein the metabolic disorder is selected from the group consisting of insulin resistance, glucose intolerance, hyperglycemia, hyperlipidemia, pre-diabetes, type 2 diabetes, and combinations thereof.

18. The method of claim 16, wherein the metabolic disorder comprises insulin resistance.

19. The method of claim 16, wherein the one or more markers comprises Ruminococcus gnavus.

20. The method of claim 16 further comprising the step of administering a therapeutically effective amount of a trace amine-associated receptor 1 (TAAR1) inhibitor to the subject.

21. The method of claim 20, wherein the TAAR1 inhibitor is a compound of Formula 1: 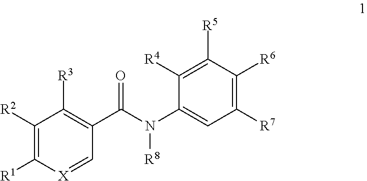
or a pharmaceutically acceptable salt thereof, wherein
n is 0, 1, 2 or 3;
p is 0 or 1;
R¹ is hydrogen, halogen, C₁-C₆ alkyl, C₁-C₆ alkyl substituted by halogen, cycloalkyl, C₁-C₆ alkoxy, NO₂, —(CH₂)ₚS(O)₂R, phenyl, morpholin-4-yl, pyrrolidin-1-yl, pyrazol-1-yl, piperidin-1-yl, 4-methyl-piperidin-1-yl, 4-cyano-piperidin-1-yl, 4-trifluoromethyl-piperidin 1-yl, piperazin-1-yl, 4-methyl-piperazin-1-yl, 3,5-dimethyl-piperidin-1-yl, piperazin-1-yl substituted by C(O)O—C₁-C₆ alkyl, 1,1-dioxoisothiazolidin-2-yl, azepan-1-yl, azetidin-1-yl, 5,6-dihydro-4H-pyran-2-yl-, tetrahydro-pyran-2-yl, NR′R″ or C(O)CF₃;
R² is hydrogen, halogen, C₁-C₆ alkyl, C₁-C₆ alkoxy, C₁-C₆ alkyl substituted by halogen, C₁-C₆ alkoxy substituted by halogen, cyano, NO₂, —(CH₂)ₚS(O)₂R, —OS(O)₂NR′R″, C₁-C₆ alkyl-O—C(═CH₂)—, —C(O)—C₁-C₆ alkyl, tetrahydro-furan-2-yl, morpholin-4-yl, pyrazol-1-yl, or —OC(O)—C₁-C₆ alkyl; or R¹ and R² together with the corresponding C-atoms form a ring comprising —CH═CH—CH═CH— or —S—(CH₂)₄—;
R³ is hydrogen, halogen, C₁-C₆ alkyl or C₁-C₆ alkoxy;
R⁴ is hydrogen, C₁-C₆ alkoxy or halogen;
R⁵ and R⁷ are each independently hydrogen, halogen, C₁-C₆ alkyl, C₁-C₆ alkoxy, NO₂, cyano, C₁-C₆ alkyl substituted by halogen, C₁-C₆ alkoxy substituted by halogen, phenyl, O-phenyl, —(CH₂)ₚS(O)₂R, NHC(O)—C₁-C₆ alkyl, C(O)—C₁-C₆ alkyl, C(O)O—C₁-C₆ alkyl or 2,5-dimethyl-imidazol-1-yl-methyl;
R⁶ is hydrogen, C₁-C₆ alkoxy, cyano, nitro, C₁-C₆ alkyl, phenyl, C₁-C₆ alkyl substituted by halogen, C₁-C₆ alkoxy substituted by halogen, C(O)O—C₁-C₆ alkyl, C(O)O—(CH₂)₂—NR′R″, oxazol-5-yl or halogen; or R⁵ and R⁶ form together with the corresponding C-atoms a ring comprising —CH═CH—CH═CH—;
R⁸ is hydrogen or C₁-C₆ alkyl;
X is —C(R⁹)═ or —N═;
R⁹ is hydrogen, C₁-C₆ alkoxy, NO₂, or halogen;
R is C₁-C₆ alkyl, morpholin-4-yl, pyrrolidin-1-yl, phenyl optionally substituted by halogen, CH₂CN, NR′R″, piperidin-1-yl, piperazin-1-yl, 3,5-dimethyl-piperidin-1-yl, azetidin-1-yl or azepane-1-yl; and
R′ and R″ are each independently hydrogen, C₁-C₆ alkyl, (CH₂)ₙ-4-methylpiperidin-1-yl, (CH₂)ₙ—C(O)—C₁-C₆ alkyl, (CH₂)ₙ-phenyl optionally substituted by halogen or (CH₂)ₙ—O—C₁-C₆ alkyl.

22. The method of claim 20, wherein the TAAR1 inhibitor is N-(3-ethoxy-phenyl)-4-pyrrolidin-1-yl-3-trifluoromethyl-benzamide.

23. The method of claim 21, wherein the one or more markers comprises Ruminococcus gnavus and the metabolic disorder comprises insulin resistance.


SEQUENCE LISTING (6 sequences)

SEQ ID NO: 1 - DNA, 24 bp, Mus musculus
  source 1..24: mol_type = genomic DNA
gacaaaacgtagttggaagactga

SEQ ID NO: 2 - DNA, 25 bp, Mus musculus
  source 1..25: mol_type = genomic DNA
gtgtgcctagaaaccttaacatctg

SEQ ID NO: 3 - DNA, 24 bp, Mus musculus
  source 1..24: mol_type = genomic DNA
aatgtttgtgatagcgtggcaaag

SEQ ID NO: 4 - DNA, 22 bp, Ruminococcus gnavus
  source 1..22: mol_type = genomic DNA
cggtcctcgggatatgataaga

SEQ ID NO: 5 - DNA, 20 bp, Ruminococcus gnavus
  source 1..20: mol_type = genomic DNA
gaccctccgcttacaaagac

SEQ ID NO: 6 - DNA, 21 bp, Ruminococcus gnavus
  source 1..21: mol_type = genomic DNA
aggcagctgatctcaacaatg